(19) United States
(12) Patent Application Publication
(10) Pub. No.: US 2005/0164970 A1
(43) Pub. Date: Jul. 28, 2005

(54) Method for treating prostate cancer using siRNA duplex for androgen receptor

(75) Inventor: Benyi Li, Overland Park, KS (US)

Correspondence Address: STINSON MORRISON HECKER LLP;ATTN: PATENT GROUP, 1201 WALNUT STREET, SUITE 2800, KANSAS CITY, MO, 64106-2150, US

(73) Assignee: University of Kansas Medical Center

(21) Appl. No.: 11/021,159

(22) Filed: Dec. 22, 2004

Related U.S. Application Data
(60) Provisional application No. 60/531,881, filed on Dec. 22, 2003.

(51) Int. Cl. A61K 48/00; C07H 21/02

(52) U.S. Cl. 514/44; 536/23.1

(57) ABSTRACT

Interfering RNA duplexes directed to the androgen receptor associated with prostate cancer are provided. A method of treating prostate cancer using interfering RNA duplexes to mediate gene silencing is also provided.

### CROSS-REFERENCE TO RELATED APPLICATIONS

This application claims the benefit of U.S. Provisional Application No. 60/531,881 filed Dec. 22, 2003, which is hereby incorporated by reference.

### STATEMENT REGARDING FEDERALLY SPONSORED RESEARCH OR DEVELOPMENT

Not Applicable.

### BACKGROUND OF THE INVENTION

1. Field of the Invention

The present invention is directed to a method of treating prostate cancer using interfering RNA duplexes to mediate gene silencing.

2. Description of Related Art

Prostate cancer is a significant risk for men in the United States. Sixty years ago, it was found that androgens were required for prostate epithelial cells to proliferate, differentiate, and survive. In addition, apoptotic cell death has been found in the prostate after androgen withdrawal.

Because of this insight, androgen ablation has been widely accepted as a major medical treatment for metastatic prostate cancer. However, most patients treated by androgen ablation ultimately relapse to more aggressive incurable androgen-refractory prostate cancer.

Anti-androgen withdrawal syndrome is another concern for androgen antagonist therapy. The etiology of androgen-independent relapse may have various molecular causes, but in each scenario, the androgen receptor (“AR”) is expressed and its function is maintained, suggesting that androgen-independent AR signaling is involved. In a transgenic mouse model, AR overexpression in prostate epithelium resulted in marked increases in epithelial proliferation and focal areas of intraepithelial neoplasia in the ventral prostate and dorsolateral prostate. Recently, the critical role of the AR for cellular proliferation in vitro or tumor growth in vivo of prostate cancer has been demonstrated by several different approaches, including disruption of AR function by anti-AR antibody and the reduction of AR expression by AR specific ribozyme or antisense oligonucleotides (Zegarra-Moro 2002, Eder 2000, Eder 2002). However, the role of the AR in cellular survival remains unknown in prostate cancer.

Apoptosis, or programmed cell death, is a well-conserved process whose basic tenets remain common to all metazoans (Hengartner 2000, Danial 2004). Intracellular organelles, like mitochondria, are key participants in apoptosis. The main aspects of mitochondrial involvement in apoptotic process include two critical events: (1) the release of mitochondrial proteins, including cytochrome c and (2) the onset of multiple parameters of mitochondrial dysfunction, such as loss of membrane potential. The Bcl-2 family proteins are critical regulators that directly control the mitochondria function and consist of both pro-apoptotic and anti-apoptotic members (Boise 1993). Bax, Bak, and Bok are pro-apoptotic members, as are the BH3-domain only members such as Bad, Bik, and Bid. Anti-apoptotic members include Bcl-2 and Bcl-x_{L}, Bcl-w, and Mcl-1. It is believed that the relative levels of pro-apoptotic and anti-apoptotic members are the key determinants in the regulation of cell death and survival.

The bcl-x gene encodes multiple spliced mRNAs, of which Bcl-x_{L} is the major transcript (Boise 1993, Gonzalez-Garcia 1994). Like Bcl-2, Bcl-x_{L} protects cells from apoptosis by regulating mitochondria membrane potential and volume, and subsequently prevents the release of cytochrome c and other mitochondrial factors from the intermembrane space into cytosol. In addition, Bcl-x_{L} may prevent apoptosis via a cytochrome c-independent pathway (Li, F. 1997). Although Bcl-x_{L} protein can be regulated post-transcriptionally, it is mainly controlled at the gene expression level (Grad 2000). Bcl-x_{L} protein is detected in the epithelial cells of normal prostate gland and prostate cancers and the expression level of Bcl-x_{L} protein correlated with higher grade and stage of the disease, indicating an important role of Bcl-x_{L} in prostate cancer progression (Krajewska 1996).

RNA interference (“RNAi”) is a recently discovered mechanism of post-transcriptional gene silencing in which double-stranded RNA corresponding to a gene (or coding region) of interest is introduced into an organism, resulting in degradation of the corresponding mRNA. The phenomenon was originally discovered in Caenorhabditis elegans by Fire and Mello.

Unlike antisense technology, the RNAi phenomenon persists for multiple cell divisions before gene expression is regained. The process occurs in at least two steps: an endogenous ribonuclease cleaves the longer dsRNA into shorter, 21- 22- or 23-nucleotide-long RNAs, termed “small interfering RNAs” or siRNAs (Hannon 2002). The siRNA segments then mediate the degradation of the target mRNA. RNAi has been used for gene function determination in a manner similar to but more efficient than antisense oligonucleotides. By making targeted knockouts at the RNA level by RNAi, rather than at the DNA level using conventional gene knockout technology, a vast number of genes can be assayed quickly and efficiently. RNAi is therefore an extremely powerful, simple method for assaying gene function.

RNA interference has been shown to be effective in cultured mammalian cells. In most methods described to date, RNA interference is carried out by introducing double-stranded RNA into cells by microinjection or by soaking cultured cells in a solution of double-stranded RNA, as well as transfecting the cells with a plasmid carrying a hairpin-structured siRNA expressing cassette under the control of suitable promoters, such as the U6, H1 or cytomegalovirus (“CMV”) promoter (Sui 2002, Paddison 2002, Yu 2002, Zia 2002, Brummelkamp 2002, Harborth 2001, Elbashir 2001, Miyagishi 2002, Lee 2001, Paul 2002). The gene-specific inhibition of gene expression by double-stranded ribonucleic acid is generally described in Fire et al., U.S. Pat. No. 6,506,559, which is incorporated by reference. Exemplary use of siRNA technology is further described in McSwiggen, Published U.S. Patent Application No. 2003/01090635 and Reich et al., Published U.S. Patent Application No. 20040248174, which are incorporated by reference.

### BRIEF SUMMARY OF THE INVENTION

An object of the present invention is to develop a gene therapeutic strategy for treating prostate cancer.

Another object of the present invention is to provide a method for treating cancer which results in apoptotic cell death.

Another object of the present invention is to use the RNA interference technique to achieve a profound AR gene silencing in prostate cancer cells that subsequently leads to apoptosis as evidenced by increased caspase-3 activation.

Yet another object of the present invention is to use the RNA interference technique to achieve a profound AR gene silencing in prostate cancer cells that subsequently leads to apoptosis as evidenced by increased poly (ADP)-ribose polymer (PARP) cleavage.

Yet another object of the present invention is to use the RNA interference technique to achieve a profound AR gene silencing in prostate cancer cells that subsequently leads to apoptosis as evidenced by a reduction of the anti-apoptotic protein Bcl-x_{L}.

Additional aspects of the invention, together with the advantages and novel features appurtenant thereto, will be set forth in part in the description which follows, and in part will become apparent to those skilled in the art upon examination of the following, or may be learned from the practice of the invention. The objects and advantages of the invention may be realized and attained by means of the instrumentalities and combinations particularly pointed out in the appended claims.

### BRIEF DESCRIPTION OF THE DRAWINGS

FIG. 1A is a Western blot of LNCaP human prostate cancer cells that were transfected with several siRNA oligonucleotides (1.0 nM in the media) with OLIGOFECTAMINE (Invitrogen). AR protein levels were determined about 48 hours later with AR antibodies (clone 441, Santa Cruz Biotechnology, Inc.). Actin blotting served as a loading control.

FIG. 1B is a Western blot of LNCaP cells following transfection with the siRNA duplexes having SEQ. ID NO. 8 and SEQ. ID NO. 31 (final concentration at about 1.0 nM). The cells were harvested 48 hours later and the AR protein expression was determined by Western blot. Actin blotting served as loading control. The siRNA was omitted in the mock control.

FIG. 2 is a Western blot of LNCaP and PC-3/AR human prostate cancer cells after the cells were transfected with different amount of the SEQ. ID NO. 31 siRNA oligonucleotide in the media with OLIGOFECTAMINE. The AR protein levels were determined 48 hours later by Western blot with AR antibodies (clone 441 Santa Cruz Biotechnology, Inc.). Actin blotting served as a loading control.

FIG. 3A is a Western blot of androgen-refractory LNCaP-Rf cells transfected with the siRNA oligonucleotides having SEQ. ID NOS. 8 and 31 with OLIGOFECTAMINE. Mock transfection was performed by omitting the siRNA. Protein levels of AR, human glycogen synthase kinase 3β (“GSK-3β”), and actin were assessed three days later. Antibodies were obtained from Santa Cruz Biotechnology, Inc.

FIG. 3B is an immunostaining showing the results of the siRNA SEQ. ID NO. 31 in cells were counterstained with propidium iodide (“PI”), a fluorescent dye for nucleotide acid staining. The LAPC-4 cells were transfected with the siRNA duplex (10 nM in the media) as indicated for 78 hours, and then subjected to immunofluorescent staining.

FIGS. 4A-C show that AR silencing leads to cell death in both LNCaP, C4-2, and LAPC-4 human prostate cell lines. In FIG. 4A-C, cells seeded in 6-well plates were transfected with the siRNAs (10 nM in the media) as indicated. Cell survival rate was determined in each time point by trypan blue exclusion assay.

FIG. 5 shows the survival rate of three cell lines that were seeded in 35-mm dishes at a density of about 10³ cells per dish overnight and then transfected with the siRNA duplexes (10 nM in the media). Mock transfection was done by omitting siRNA. The clonogenic survival fraction of the cells was determined at seven days post-transfection. Colonies were fixed, stained, and photographed. The clonogenic survival rate in control group was designated as 100%. Data represents three different experiments.

FIG. 6 shows LNCaP-Rf cells growing in RPMI media supplied with 5% charcoal-stripped fetal bovine serum (“cFBS”) transfected with the siRNA oligonucleotides as indicated with OLIGOFECTAMINE (panels f-h). Control cells received nothing (panel a), the OLIGOFECTIMINE only (panel e), or the siRNA oligonucleotides (panels b-d). The photographs were taken seven days later on an inverted microscope.

FIGS. 7 A&B are photographs providing visualization of the fluorescent dye Cy3-labled AR siRNA induced cell death. In FIG. 7A, LNCaP cells were seeded in 6-well plates overnight and then transfected with the Cy3-labled siRNAs (10 nM in the media) as indicated and cell death was monitored daily. Pictures were taken at day 1 and day 4 after transfection. The Cy3-labeled siRNAs are seen as white dots in Cy3 panels (b, d, f, h). In panels g and h, white arrows indicate several living cells without the Cy3 -labeling (negative transfection) while a black arrow indicates a cluster of dying cells (detached) with strong Cy3-labeling (positive transfection). In FIG. 7B, LNCaP cells were monitored for five days before the pictures were taken after transfection with the siRNA duplexes plus the green fluorescent protein pGFPhAR construct. In panels c-d, white arrow indicates a living cell that maintains green fluorescent protein (“GFP”) expression.

FIG. 8 shows that siRNA-mediated AR silencing leads to apoptosis. FIG. 8A shows that after transfection with the siRNA duplexes (10 nM in the media) as indicated for four days, LNCaP cells were harvested for measuring the apoptotic cell death using an Annexin V-FITC kit. The data represents two different experiments. In FIG. 8B, following transfection with the siRNA duplexes (10 nM in the media) for five days, LNCaP cells were harvested and lysed to determine the proteolytic process of caspase-3 and caspase-8, poly (ADP)-ribose polymer (“PARP”) cleavage, as well as the expression levels of Bcl-2, Bcl-xL, Bax, and Bak by a Western blot assay.

FIGS. 8C&D is a Western Blot of LNCaP cells after seven days of transfection with the siRNAs as indicated. The LNCaP cells were harvested and the cytosolic occurrence of cytochrome c, proteolytic process of caspase-3 and caspase-6, DFF45, and PARP cleavage were determined by Western blot.

FIG. 8E graphically illustrates the fold induction after seven days of transfection with the siRNAs as indicated. The LNCaP cells were washed with ice-cold PBS and then harvested. Caspase activity was measured using a commercially available APO-ONE Homogeneous Caspase-3/7 Assay kit. The mean value of the relative activity was shown from three independent experiments.

FIG. 8F is a photograph taken following transfection with the siRNA duplexes (10 nM in the media) for five days. The LNCaP cells were incubated with the fluorescent cationic dye JC-1 (about 0.3 μg/ml) for about 15 minutes at about 37° C. The pictures were taken under a fluorescent microscope (magnitude×40). Data was reproducible in three independent experiments.

FIG. 9A is a Western blot of LNCaP cells after serum starvation for 24 hours. The LNCaP cells were treated with R1881 (metribolone, a synthetic androgen) in the present or absent of antiandrogen bicalutamide for another 24 hours. Cells were harvested and Bcl-x_{L} protein level was determined by Western blot, and actin blot served as loading control.

FIG. 9B graphically illustrates the results after LNCaP cells were co-transfected with a luciferase reporter construct Bcl-x_{L}-LUC together with an internal control reporter construct pCMV-SEAP overnight and then the cells were serum-starved for 24 hours. The solvent ethanol (control), R1881 in different doses as indicated, or insulin-like growth factor-1 (“IGF-1”) (10 ng/ml) alone was added once in the culture media containing 2% cFBS for another 24 hours (left-half panel) or for a different time period as indicated (right-half panel). Luciferase or secreted alkaline phosphatase (“SEAP”) activities were measured, and the luciferase activity was presented as fold induction against control sample after normalized with protein content and SEAP activity.

FIG. 9C shows a chromatin immunoprecipitation (“ChIP”) assay after LNCaP cells were serum-starved for 24 hours and then untreated or treated with R1881 (1.0 nM) for 18 hours in the presence or absence of the antiandrogen bicalutamide (10 μM). Binding of AR to the bcl-x promoter was determined with the CHIP assay (lanes 7-9). As controls, sample lysates were also incubated with a normal rabbit serum IgG (lanes 4 and 6). Lanes 1 and 3 represent input signals obtained from 1% input chromatin IP Ab, immunoprecipitation antibody. Data represent three independent experiments.

FIG. 9D is a reverse transcription polymerase chain reaction (“RT-PCR”) assay and Western blot following transfection with the siRNA duplexes (final concentration at 10 nM in the medium) as indicated, in which LNCaP cells were harvested 72 hours later. The mRNA levels of target genes as indicated were determined by RT-PCR assay (upper panel), and the AR protein was determined by Western blot (bottom panel) and actin blot served as loading control. The siRNA was omitted in the mock control.

FIG. 9E shows the RT-PCR assay results of LNCaP cells transfected with different AR siRNA (as indicated) and then harvested at 72 hours later. The MRNA levels of the bcl-x gene as indicated were determined by RT-PCR assay. The 28S gene served as internal control.

FIGS. 9F&G is a Western Blot of LNCaP cells after transfection with siRNA SEQ. ID NO. 8 or negative control siRNA (10 nM in the medium) as indicated. The LNCaP cells were harvested at each time point (FIG. 9F) or day 7 (FIG. 9G), and the protein levels of AR, Bcl-2, Bcl-x_{L}, Bax, Bak and XIAP were assessed by Western blot. Data was reproducible in three independent experiments.

FIG. 9H shows the results of LNCaP/Puro and LNCaP/Bcl-x_{L} cells after being transfected with AR siRNA SEQ. ID NO. 8 for seven days and the expression level of endogenous/exogenous bcl-xl gene determined by Western blot. The membrane was reprobed with anti-HA antibody to show the exogenous Bcl-x_{L} protein. Actin blot served as loading control. The relative cell death rate was determined by trypan blue exclusion assay. The asterisk indicates a significant difference (P<0.05) between LNCaP/Puro vs LNCaP/Bcl-xL cells after the siRNA having SEQ. ID NO. 8 transfection. Data represent three independent experiments.

FIG. 91 (upper panel) shows the parental LNCaP cells (lane 1), LNCaP subclone LN #11 (lane 2) and a stable subclone bearing an empty vector (lane 3) after being exponentially grown and harvested. Total RNA was isolated from Bcl-x_{L} mRNA levels were determined by RT-PCR, and 28S gene served as internal control for the RT-PCR assay. Cellular proteins were extracted and Bcl-x_{L} protein levels were assessed by Western blot, and anti-actin blot served as loading control. The data represent two separate experiments. In the lower panel, the cells were transfected with negative siRNA (black column) or AR siRNA SEQ. ID NO. 8 (shaded column) at 10 nM in the culture medium supplied with 2% cFBS. Cell death rate was determined five days later by trypan blue exclusion assay. The asterisk indicates a significant difference (P<0.05) between LNCaP subclone LN#11 vs. LNCaP cells.

FIG. 10 (upper panel) shows the Western blot of cells harvested from the experiments described in FIG. 34C after being lysed to determine the protein levels of the AR. Actin blot served as loading control. In the lower panel, three prostate cell lines (RWPE-1, LAPC-4 and 22Rv1) were transfected with AR siRNA SEQ. ID NO. 8 (black column) or negative siRNA (shaded column) at 10 nM in the culture medium supplied with 2% cFBS, and cell survival rate was determined seven days later by trypan blue exclusion assay. Mock transfection was made by omission of the siRNA (open column).

FIG. 11A illustrates the AR hairpin constructed by linking the sense and antisense sequence of the siRNA having SEQ. ID NO. 8.

FIG. 11B illustrates the AR responsive reporter Probasin-secreted alkaline phosphatase (“SEAP”) transfected with or without the pU6-ARHP8 into LNCaP cells followed by serum starvation for 34 hours. After addition of R1881 (1.0 nM) or FGF-2 (10 ng/ml) for 24 hours, the culture media were collected and SEAP activity was measured.

FIG. 11C illustrates the culture in which a construct bearing a fusion protein of AR and green fluorescent protein (“GFP”) was transfected with (panels c&d) or without (panels a&b) the pU6-ARHP8 into LNCaP cells. The pictures were taken 72 hours later.

FIG. 12A illustrates the scheme of construct generation of pU6ARHP-CX1GFP.

FIGS. 12B & C show cells transfected with the pCX1-eGFP plasmid by CYTOFECTENE reagent (BioRad) and GFP expression was evaluated 24 hours later under a fluorescent microscope.

FIG. 13 is a photograph showing the results of recombinant adeno-associated virus (“AAV”) infection in prostate cancer PC-3 (panels a and b) and LNCaP (panels c and d) cells. The cells were infected with the recombinant adeno-associated virus (“rAAV”) carrying alkaline phosphatase (“AP”) gene (panel a), LacZ (panel c), or mock-infected (panels b, d). Transgene expression was evaluated five days later by cytochemical staining for AP (panels a and b) or LacZ (panels c and d) activity.

FIG. 14 illustrates the experimental design to test for siRNA mediated AR gene silencing in prostate cancer xenograph mouse model.

FIG. 15 shows the experimental design to test for siRNA AR gene silencing on acquisition of the androgen-independent phenotype by prostate cancer cells in vivo.

FIG. 16 illustrates the experimental design to evaluate the effect of siRNA AR gene silencing on tumor growth of prostate cancer xenograft from androgen-independent cell lines.

### DETAILED DESCRIPTION OF THE INVENTION

The AR has been shown to play a critical role in androgen-independent progression of prostate cancer. The present invention is directed to a novel method of targeting the AR gene by knocking down or inhibiting its expression as a novel strategy for prostate cancer therapy. The present invention includes compositions and methods comprising siRNA targeted to AR mRNA, which are advantageously used to inhibit prostate cancer. The siRNA of the invention are believed to cause the RNAi-mediated degradation of these mRNAs so that the protein products of the AR gene is not produced or are produced in reduced amounts.

The invention therefore provides isolated siRNA comprising short double-stranded RNA from that are targeted to the target MRNA. The siRNA's comprise a sense RNA strand and a complementary antisense RNA strand annealed together by standard Watson-Crick base-pairing interactions (hereinafter “base-paired”). Preferably, the sense strand comprises a nucleic acid sequence which is substantially identical to a target sequence contained within the target mRNA.

As used herein, a nucleic acid sequence “substantially identical” to a target sequence contained within the target MRNA is a nucleic acid sequence which is identical to the target sequence, or which differs from the target sequence by one or more nucleotides. Sense strands of the invention which comprise nucleic acid sequences substantially identical to a target sequence are characterized in that siRNA comprising such sense strands induce RNAi-mediated degradation of mRNA containing the target sequence. For example, an siRNA of the invention can comprise a sense strand comprise nucleic acid sequences which differ from a target sequence by one, two or three or more nucleotides, as long as RNAi-mediated degradation of the target mRNA is induced by the siRNA.

The sense and antisense strands of the present siRNA can comprise two complementary, single-stranded RNA molecules or can comprise a single molecule in which two complementary portions are base-paired and are covalently linked by a single-stranded “hairpin” area. That is, the sense region and antisense region can be covalently connected via a linker molecule. The linker molecule can be a polynucleotide or non-nucleotide linker. The siRNA can also contain alterations, substitutions or modifications of one or more ribonucleotide bases. For example, the present siRNA can be altered, substituted or modified to contain one or more deoxyribonucleotide bases.

The siRNA of the invention can comprise partially purified RNA, substantially pure RNA, synthetic RNA, or recombinantly produced RNA, as well as altered RNA that differs from naturally-occurring RNA by the addition, deletion, substitution and/or alteration of one or more nucleotides. Such alterations can include addition of non-nucleotide material, such as to the end(s) of the siRNA or to one or more internal nucleotides of the siRNA; modifications that make the siRNA resistant to nuclease digestion (e.g., the use of 2′-substituted ribonucleotides or modifications to the sugar-phosphate backbone); or the substitution of one or more nucleotides in the siRNA with deoxyribonucleotides.

The siRNA of the invention can be obtained using a number of techniques known to those of skill in the art. For example, the siRNA can be chemically synthesized or recombinantly produced using methods known in the art, such as the Drosophila in vitro system described in U.S. published application 2002/0086356 of Tuschl et al., the entire disclosure of which is herein incorporated by reference. The siRNA of the invention may be chemically synthesized using appropriately protected ribonucleoside phosphoramidites and a conventional DNA/RNA synthesizer. The siRNA can be synthesized as two separate, complementary RNA molecules, or as a single RNA molecule with two complementary regions. Commercial suppliers of synthetic RNA molecules or synthesis reagents include Proligo (Hamburg, Germany), Dharmacon Research (Lafayette, Colo., USA), Pierce Chemical (part of Perbio Science, Rockford, Ill., USA), Glen Research (Sterling, Va., USA), ChemGenes (Ashland, Mass., USA) and Cruachem (Glasgow, UK).

The siRNA can also be expressed from recombinant circular or linear DNA plasmids using any suitable promoter. Suitable promoters for expressing siRNA of the invention from a plasmid include, for example, the U6 or H1 RNA pol III promoter sequences and the cytomegalovirus promoter. Selection of other suitable promoters is within the skill in the art. The recombinant plasmids of the invention can also comprise inducible or regulatable promoters for expression of the siRNA in a particular tissue or in a particular intracellular environment.

The siRNA expressed from recombinant plasmids can either be isolated from cultured cell expression systems by standard techniques, or can be expressed intracellularly. The use of recombinant plasmids to deliver siRNA of the invention to cells in vivo is discussed in more detail below. siRNA of the invention can be expressed from a recombinant plasmid either as two separate, complementary RNA molecules, or as a single RNA molecule with two complementary regions. Selection of plasmids suitable for expressing siRNA of the invention, methods for inserting nucleic acid sequences for expressing the siRNA into the plasmid, and methods of delivering the recombinant plasmid to the cells of interest are within the skill in the art.

The siRNA of the invention can also be expressed from recombinant viral vectors intracellularly in vivo. The recombinant viral vectors of the invention comprise sequences encoding the siRNA of the invention and any suitable promoter for expressing the siRNA sequences. Suitable promoters include, for example, the U6 or H1 RNA pol III promoter sequences and the cytomegalovirus promoter. Selection of other suitable promoters is within the skill in the art. The recombinant viral vectors of the invention can also comprise inducible or regulatable promoters for expression of the siRNA in a particular tissue or in a particular intracellular environment. siRNA of the invention can be expressed from a recombinant viral vector either as two separate, complementary RNA molecules, or as a single RNA molecule with two complementary regions. Any viral vector capable of accepting the coding sequences for the siRNA molecule(s) to be expressed can be used, for example vectors derived from adenovirus (AV); adeno-associated virus (AAV); retroviruses (e.g, lentiviruses (LV), Rhabdoviruses, murine leukemia virus); herpes virus, and the like. The tropism of viral vectors can be modified by pseudotyping the vectors with envelope proteins or other surface antigens from other viruses, or by substituting different viral capsid proteins, as appropriate.

The siRNA of the present invention is preferably isolated. As used herein, “isolated” means synthetic, or altered or removed from the natural state through human intervention. For example, a siRNA naturally present in a living animal is not “isolated,” but a synthetic siRNA, or a siRNA partially or completely separated from the coexisting materials of its natural state is “isolated.” An isolated siRNA can exist in substantially purified form, or can exist in a non-native environment such as, for example, a cell into which the siRNA has been delivered. By way of example, siRNA which are produced inside a cell by natural processes, but which are produced from an “isolated” precursor molecule, are themselves “isolated” molecules. Thus, an isolated dsRNA can be introduced into a target cell, where it is processed by the Dicer protein (or its equivalent) into isolated siRNA.

As used herein, “inhibit” means that the activity of a gene expression product or level of RNAs or equivalent RNAs encoding one or more gene products is reduced below that observed in the absence of the nucleic acid molecule of the invention. The inhibition with a siRNA molecule preferably is below that level observed in the presence of an inactive or attenuated molecule that is unable to mediate an RNAi response. Inhibition of gene expression with the siRNA molecule of the instant invention is preferably greater in the presence of the siRNA molecule than in its absence.

As used herein, the terms “gene” or “target gene” mean a nucleic acid that encodes an RNA, for example, nucleic acid sequences including, but not limited to, structural genes encoding a polypeptide. The target gene can be a gene derived from a cell, an endogenous gene, a transgene, or exogenous genes such as genes of a pathogen, for example a virus, which is present in the cell after infection thereof.

As used herein, the phrase “highly conserved sequence region” means a nucleotide sequence of one or more regions in a target gene does not vary significantly from one generation to the other or from one biological system to the other.

As used herein, the terms “complementarity” or “complementary” means that a nucleic acid can form hydrogen bond(s) with another nucleic acid sequence by either traditional Watson-Crick or other non-traditional types of interaction. In reference to the nucleic molecules of the present invention, the binding free energy for a nucleic acid molecule with its complementary sequence is sufficient to allow the relevant function of the nucleic acid to proceed, e.g., RNAi activity. For example, the degree of complementarity between the sense and antisense strand of the siRNA construct can be the same or different from the degree of complementarity between the antisense strand of the siRNA and the target RNA sequence. A percent complementarity indicates the percentage of contiguous residues in a nucleic acid molecule that can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). “Perfectly complementary” means that all the contiguous residues of a nucleic acid sequence will hydrogen bond with the same number of contiguous residues in a second nucleic acid sequence.

As used herein, the term “cell” is defined used in its usual biological sense, and does not refer to an entire multicellular organism, e.g., specifically does not refer to a human. The cell can be present in an organism, e.g., mammals such as humans, cows, sheep, apes, monkeys, swine, dogs, and cats. The cell can be eukaryotic (e.g., a mammalian cell). The cell can be of somatic or germ line origin, totipotent or pluripotent, dividing or non-dividing. The cell can also be derived from or can comprise a gamete or embryo, a stem cell, or a fully differentiated cell.

As used herein, the term “RNA” means a molecule comprising at least one ribonucleotide residue. By “ribonucleotide” is meant a nucleotide with a hydroxyl group at the 2′ position of a beta-D-ribo-furanose moiety. The terms include double stranded RNA, single stranded RNA, isolated RNA such as partially purified RNA, essentially pure RNA, synthetic RNA, recombinantly produced RNA, as well as altered RNA that differs from naturally occurring RNA by the addition, deletion, substitution and/or alteration of one or more nucleotides. Such alterations can include addition of non-nucleotide material, such as to the end(s) of the siRNA or internally, for example at one or more nucleotides of the RNA. Nucleotides in the RNA molecules of the instant invention can also comprise non-standard nucleotides, such as non-naturally occurring nucleotides or chemically synthesized nucleotides or deoxynucleotides. These altered RNAs can be referred to as analogs or analogs of naturally-occurring RNA.

As used herein, the term “subject” means an organism, which is a donor or recipient of explanted cells or the cells themselves. “Subject” also refers to an organism to which the nucleic acid molecules of the invention can be administered. In one embodiment, a subject is a mammal or mammalian cells. In another embodiment, a subject is a human or human cells

As used herein, the term “vector” means any nucleic acid- and/or viral-based technique used to deliver a desired nucleic acid.

The following examples further illustrate the present invention in detail but are not to be construed to limit the scope thereof.

Materials and Methods

1. Cells and Reagents.

The human prostate cancer LNCaP, LAPC-4, PC-3, C4-2 and 22Rv1 cells and HEK293 cells were described previously (Liao & Thrasher 2003, Liao & Zhang 2003, Liao 2004). Prostate epithelial cell RWPE-1 and breast cancer cell lines (MCF-7 and T47D) were obtained from American Type Culture Collection (“ATCC”) (Manassas, Va.). The hormone-refractory prostate cancer cell LNCaP-Rf was a kind gift provided by Dr. Donald Tindall of the May Clinic (Zegarra-Moro 2002), and LNCaP C4-2 (Wu 1994) was obtained from UroCor, Inc. (Oklahoma City, Okla.). PC-3/AR subline was established by stably transfecting the AR-null PC-3 cells (obtained from ATCC, Manassas, Va.) with a vector bearing the human AR gene obtained from Dr. Fahri Saatcioglu. PC-3/Neo was established when an empty vector was used. The stable clones were selected in G418 and maintained in RPMI 1640 supplemented with 10% fetal bovine serum (“FBS”). The plasmid pGFP-hAR bearing a GFP-fused human AR gene was obtained from Dr. Craig Robson. LNCaP/Bcl-x_{L} subline was established by stably transfecting the LNCaP cells with a vector bearing the human bcl-xl cDNA sequence with a HA-tag obtained from Dr. Hong-gang Wang, and LNCaP/Puro was established when an empty vector was used. The stable clones were selected in a puromycine-containing culture medium. Antibodies against human AR (monoclonal), actin, and secondary antibodies were purchased from Santa Cruz Biotech (Santa Cruz, Calif.). Antibodies against caspases, cytochrome c, Bcl-2 family members, PARP, x-linked inhibitor of apoptosis protein (“XIAP”), DFF45 and PARP were obtained from Cell Signaling (Beverly, Mass.). JC-1 fluorescent dye was obtained from Molecular Probes (Eugene, Oreg.). Other reagents were supplied by Sigma (Saint Louis, Mo.). Charcoal-stripped fetal bovine serum (“cFBS”) was obtained from Atlanta Biologicals (Norcross, Ga.).

2. siRNA Synthesis, Labeling and Transfection.

Sequence information regarding the human AR gene (GenBank accession NM_{—}000044) was extracted from the NCBI Entrez nucleotide database. Up to 34 mRNA segments were identified using the OLIGOENGINE software (OligoEngine Inc., Seattle, Wash.) which fulfill the requirements for potentially triggering RNAi according to the literature (Elhashir 2000). Thirty-four sequences, which set forth the sequence for one strand of the double stranded RNA, were generated. These included the following nucleotide sequences:

| | |
|---|---|
| SEQ. ID NO. 1: | 577-cuccuucagcaacagcagc-595 |
| SEQ. ID NO. 2: | 589-cagcagcaggaagcaguau-607 |
| SEQ. ID NO. 3: | 601-gcaguauccgaaggcagca-619 |
| SEQ. ID NO. 4: | 705-cgccaaggaguuguguaag-723 |
| SEQ. ID NO. 5: | 711-ggaguuguguaaggcagug-729 |
| SEQ. ID NO. 6: | 873-agguucucugcuagacgac-891 |
| SEQ. ID NO. 7: | 874-gguucucugcuagacgaca-892 |
| SEQ. ID NO. 8: | 1324-gaaggccaguuguauggac-1342 |
| SEQ. ID NO. 9: | 1330-ggccaguuguauggaccgu-1348 |
| SEQ. ID NO. 10: | 1674-gaccugccugagcugugga-1692 |
| SEQ. ID NO. 11: | 1773-acagaaguaccugugcgcc--1791 |
| SEQ. ID NO. 12: | 1774-cagaaguaccugugcgcca-1792 |
| SEQ. ID NO. 13: | 1917-acuacaggaggaaggagag-1935 |
| SEQ. ID NO. 14: | 1918-cuacaggaggaaggagagg-1936 |
| SEQ. ID NO. 15: | 1970-cccagaagcugacaguguc-1988 |
| SEQ. ID NO. 16: | 1999-ggcuaugaaugucagccca-2017 |
| SEQ. ID NO. 17: | 2028-uguccuggaagccauugag-2046 |
| SEQ. ID NO. 18: | 2038-gccauugagccagguguag-2056 |
| SEQ. ID NO. 19: | 2076-caaccagcccgacuccuuu-2094 |
| SEQ. ID NO. 20: | 2184-cuuacacguggacgaccag-2202 |
| SEQ. ID NO. 21: | 2271-ugucaacuccaggaugcuc-2289 |
| SEQ. ID NO. 22: | 2277-cuccaggaugcucuacuuc-2295 |
| SEQ. ID NO. 23: | 2316-ugaguaccgcaugcacaag-2334 |
| SEQ. ID NO. 24: | 2363-ugaggcaccucucucaaga-2381 |
| SEQ. ID NO. 25: | 2398-aucaccccccaggaauucc-2416 |
| SEQ. ID NO. 26: | 2399-ucaccccccaggaauuccu-2417 |
| SEQ. ID NO. 27: | 2427-agcacugcuacucuucagc-2445 |
| SEQ. ID NO. 28: | 2428-gcacugcuacucuucagca-2446 |
| SEQ. ID NO. 29: | 2548-aucccacauccugcucaag-2566 |
| SEQ. ID NO. 30: | 2547-ucccacauccugcucaaga-2565 |
| SEQ. ID NO. 31: | 2564-gacgcuucuaccagcucac-2582 |
| SEQ. ID NO. 32: | 2652-gucacacauggugagcgug-2670 |
| SEQ. ID NO. 33: | 2710-gugcccaugauccuuucug-2728 |
| SEQ. ID NO. 34: | 2739-gcccaucuauuuccacacc-2757 |

The AR gene specificity was confirmed by searching NCBI BlastN database. The two segments, designated as No. 8 (SEQ. ID NO. 8: 1324-GAA GGC CAG UUG UAU GGA C-1342) and No. 31 (SEQ. ID NO. 31: 2564-GAC GCU UCU ACC AGC UCA C-2582) were selected for next experiments since they induced the most profound AR silencing compared to other segments tested in the preliminary analyses. The siRNAs were prepared by a transcription-based method using the SILENCER siRNA construction kit (Ambion, Austin, Tex.) according to the manufacturer's instructions. The 29-mer sense and antisense DNA oligonucleotide templates (Gross 1999) nucleotides specific to the targets and eight nucleotides specific to T7 promoter primer sequence 5′-CCTGTCTC-3′) were synthesized by IDT (Coralville, Iowa). The quality of the synthesized siRNA was estimated by agarose gel analysis and found to be very clean. RNAs were quantified by using RiboGreen fluorescence (Molecular Probes). A SILENCER siRNA labeling kit using a fluorescent Cy3 dye (Ambion Inc., Austin Tex.) was used for labeling the siRNA duplexes according to the manufacturer's instructions.

All of the thirty-four purified siRNA duplexes were transfected into LNCaP cells with the OLIGOFECTAMINE reagent (Invitrogen Co., Carlsbad, Calif.) in a medium supplied with 2% charcoal-stripped fetal bovine serum. The media were changed every three days. A scrambled negative siRNA duplex (Ambion Inc.) was used as control. A pooled chemically synthesized AR siRNA mixture was purchased from Upstate Group, Inc., (Charlottesville, Va.) for use in the Examples.

3. Cytotoxicity Assays and Flow Cytometry.

Typically, cell viability was assessed with a trypan blue exclusion assay (Liao 2003). For clonogenic survival assay, about 10³ cells were seeded in a 35-mm dish and transfected with the siRNAs. The media were changed every three days and the cultures were observed daily for colony formation. On day seven, the cultures were washed with phosphate-buffered saline (“PBS”), fixed, and stained as previously described (Tosetti 2003). The colonies were counted under an inverted microscope. Apoptotic cell death was determined using an Annexin V-FITC Apoptosis Detection Kit (BD PharMingen, San Diego, Calif.) according to the manufacturer's manual. Briefly, cells were harvested and washed with ice-cold PBS and then suspended in Annexin V binding buffer. Then, cells were stained for about 15 minutes at room temperature in the dark and analyzed on a FACS Calibur flow cytometer using CELLQuest software.

4. Western Blotting and Immunofluorescence.

For the Western blots, cells were washed in PBS and lysed in a RIPA buffer supplied with protease inhibitors (CytoSignal, Irvine, Calif.). A Western blot analysis was performed as described previously (Li 2000) to assess the protein expression level of target molecules, such as AR, actin, caspase-3, caspase-8, Bcl-2 family members, and PARP. The blots were developed with a SuperSignal West Dura Substrate kit (Pierce Biotech, Rockford, Ill.). Immunofluorescent staining was performed as previously described (Li 2000). The pictures were taken under a fluorescence microscope (Nikon) set at 100× magnification in Example 2.

5. mRNA expression analysis and RT-PCR.

Total RNA was prepared using TRIZOL reagent (Invitrogen Co., Carlsbad, Calif.). To assess mRNA expression, a semiquantitative reverse transcription-PCR (RT-PCR) method was used as described previously (Li 2000). RT-PCR was done using a RETROscript kit from Ambion Inc. per manufacturer's manual (Austin, Tex.). The primers and PCR conditions were described as follow: for human AR gene⁶ (SEQ. ID NO. 35: forward 5′-cctggcttccgcaacttacac-3′; (SEQ. ID NO. 36 backward 5′-ggacttgtgcatgcggtactca-3′); human PSA gene (Shariat 2002) (SEQ. ID NO. 37: forward 5′-gatgactccagccacgacct-3′; SEQ. ID NO. 38 backward 5′-cacagacaccccatcctatc-3′); human bcl-xl gene (Mercatante 2002) (SEQ. ID NO. 39: forward 5′-catggcagcagtaaagcaag-3′; SEQ. ID NO. 40 backward 5′-gcattgttcccatagagttcc-3′). 28S ribozyme RNA (SEQ. ID NO. 41: forward 5′-gttcacccactaatagggaac gtg-3′; SEQ, ID NO. 42 backward, 5′-gattctgacttagaggcgttcagt-3′) was used as an internal control (Goffin 2003). The primers were synthesized by IDT (Coralville, Iowa). The amplification profile. was as follows: 95° C. for 30 seconds, 56° C. for 30 seconds, and 72° C. for one minute running in a total of 25 cycles. After 25 amplification cycles, the expected PCR products were size fractionated onto a 2% agarose gel and stained with ethidium bromide.

6. Mitochondrial Membrane Potential, Caspase Activity and Luciferase Reporter Gene Assay.

The siRNA-transfected cells were incubated in the presence of JC-1 solution, which was added to the culture medium at a final concentration of 0.3 μg/ml, for 15 minutes at 37° C. Thereafter, the cells were analyzed under a fluorescent microscope. The caspase activity was measured using an APO-ONE Homogeneous Caspase-3/7 Assay kit obtained from Promega (Madison, Wis.) per the manufacturer's manual. Briefly, the cells were washed in ice-cold PBS and then suspended in the assay buffer containing the substrate rhodamine 110 (Z-DEVD-R110) provided by the supplier. The amount of fluorescent product generated is measured at 480/520 nM using a FLUOROSCAN fluorescent reader as described previously (Liao & Thrasher 2003, Liao & Zhang 2003, Liao 2004). For Bcl-xL reporter gene assay, a luciferase reporter plasmid controlled by the full length (3.2 kb) of the mouse bcl-xl promoter (Bcl-xL-LUC) was obtained from Dr Gabriel Nunez (Grillot 1997). A construct pCMV-SEAP was used as an internal reference control and the assay procedure were described in detail previously (Liao & Thrasher 2003, Liao & Zhang 2003, Liao 2004). The luciferase activity of each sample was normalized against the corresponding SEAP activity before the fold induction value relative to control cells was calculated.

7. Chromatin Immunoprecipitation (ChIP) Assay.

Cells were maintained in 10-cm dishes in medium without serum for at least 16 hours and treated with or without 1.0 nM R1881 for 12 hours. The ChIP assay was performed using a ChIP assay kit and the polyclonal antibody against AR were obtained from Upstate according to the manual (Charlottesville, Va.). Normal rabbit serum was used as a negative control (Santa Cruz Biotechnology). The primers for the PCR were SEQ. ID NO. 43: 5′-cgatggaggaggaagcaagc-3′ and SEQ. ID NO. 44: 5′-gcaccacctacattcaaatcc-3′, which amplify a 250-bp fragment corresponding to human bcl-x gene promoter sequence −390 to −640 from the transcription start site (GenBank accession number D30746).

8. Statistical Analysis.

All experiments were repeated two or three times. Western blot results are presented from a representative experiment. The mean and standard deviation from two experiments for cell viability are shown. The number of viable cells in the control group was assigned a relative value of 100%. The significant differences between groups were analyzed using the SPSS computer software (SPSS Inc., Chicago, Ill.).

### EXAMPLE 1

### Androgen Receptor Silencing via RNA Interference

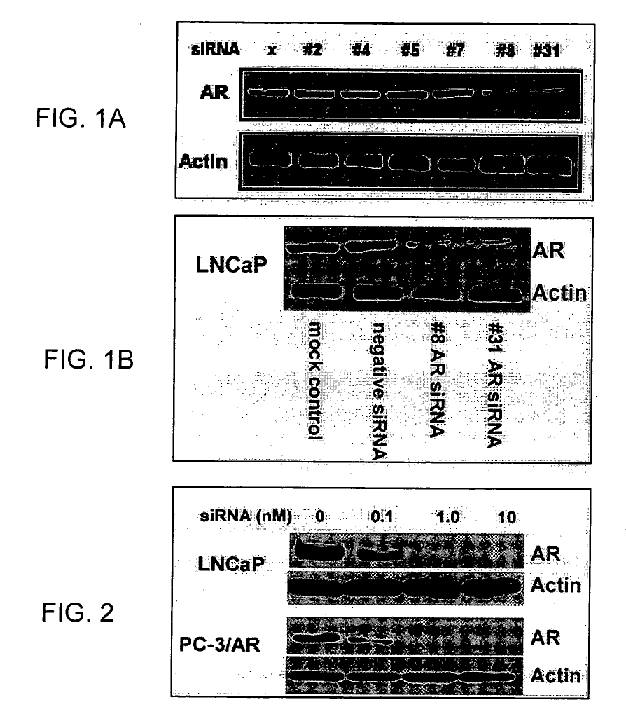

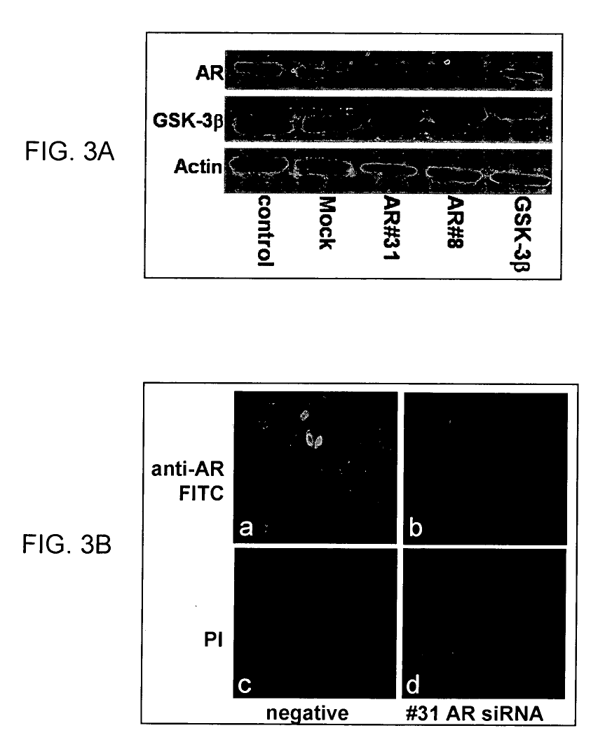

In this example, it was shown that the androgen receptor can be silenced via RNA interference in both androgen-sensitive and androgen-insensitive cells. In a preliminary analysis of a panel of siRNAs against the AR gene, two potent siRNAs were identified in knocking down or inhibiting AR expression. As shown in FIG. 1A and FIG. 1B, both of the selected siRNAs (SEQ. ID NO. 8 and SEQ. ID NO. 31) significantly knocked down AR expression at a final concentration of 1.0 nM in culture media.

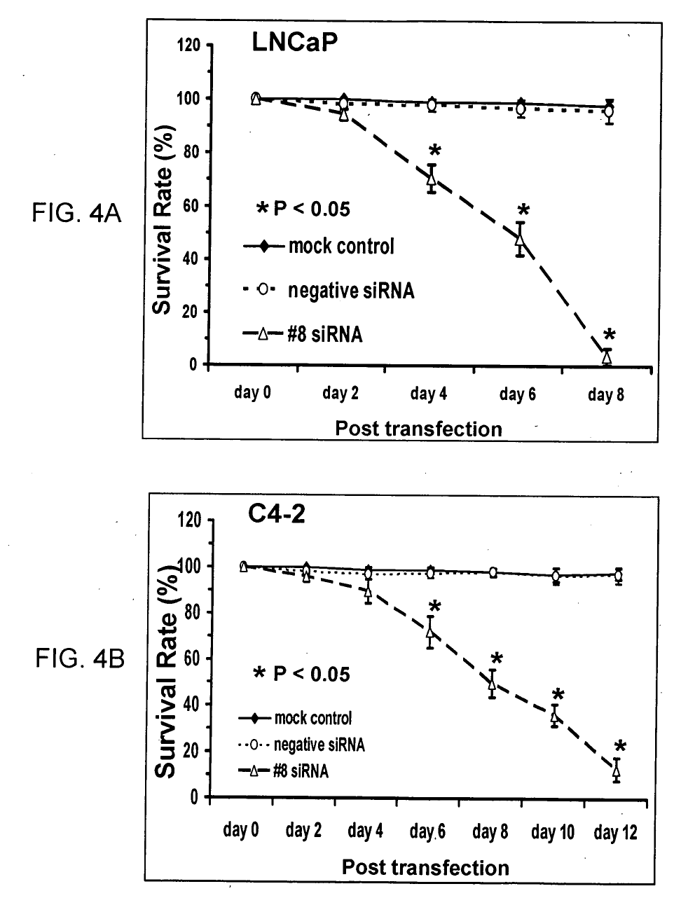

Next, three different doses of the AR siRNA having SEQ. ID NO. 31 were compared in both androgen-sensitive LNCaP and androgen-insensitive PC-3/AR cells. The PC-3 cells were obtained from the ATCC, and the subline PC-3/AR established by stable transfection of exogenous wild type AR. Actin blotting served as a loading control. As shown in FIG. 2, the AR siRNA having SEQ. ID NO. 31 reduced AR protein expression in both cell lines after 48 hours of transfection in a dose-dependent manner.

### EXAMPLE 2

### siRNA-Mediated AR Silencing Leads to Cell Death

The AR has been demonstrated to be important for cell proliferation in vitro (Zegarra-Moro 2002, Eder 2000) or tumor growth in vivo (Eder 2002) in prostate cancer. A recent report also showed a reduced cell proliferation after AR silencing by using the RNAi approach (Wright 2003). As discussed more fully below, the present invention showed for the first time that if androgen-sensitive LNCaP cells were kept in AR silencing condition for more than about 4-5 days, a significant cell death was induced in addition to cell arrest.

The effect of the siRNA transfection on cell survival of the cells was first evaluated. As a control, a siRNA against human glycogen synthase kinase 3β (“GSK-3β”) (Gene Bank #NM002093.2, SEQ. ID NO. 45 284-GAAUCGAGAGCUCCAGAUC-303 was used. The androgen-refractory cell line LNCaP-Rf (a kind gift from Dr. Donald Tindall, Mayo Clinic) was used in this example. LNCaP-Rf was established by long-term culture of LNCaP cells (approximately greater than 10 weeks) in RPMI 1640 with aboutlO% cFBS. In addition, PC-3/AR cells and PC-3/Neo cells (derived from PC-3 cells stably transfected with a vector carrying Neo gene) were also used. These cells were growing in RPMI media with regular serum.

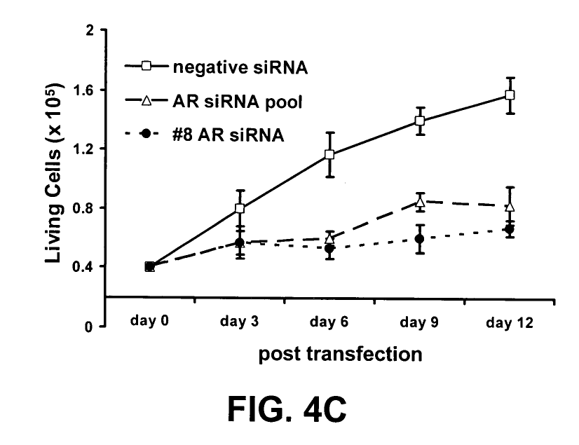

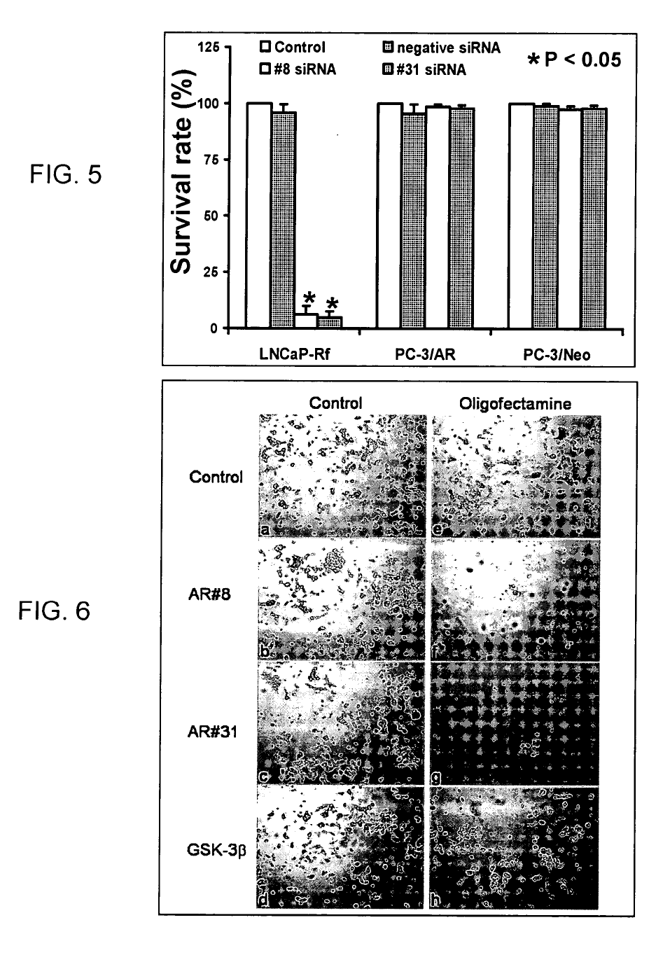

It was first determined if the AR siRNAs induce AR gene silencing in LNCaP-Rf cells. As shown in FIG. 3A, the protein levels of AR and GSK-3β were largely reduced after three days transfection of the AR siRNAs (both SEQ. ID NO. 8 and SEQ. ID NO. 31) and GSK-3β siRNA at 1.0 nM respectively, without non-specific cross-effect.

The efficiency of the siRNA-induced AR gene silencing was next evaluated by immunostaining. The cells were grown on chambered glass slides and transfected as above for three days. As shown in FIG. 3B, the AR protein is expressed mainly in the nuclear compartment of LNCaP-Rf cells, and more than 90% of the cells showed reduced AR immunostaining after transfection with the AR siRNA oligonucleotides (e.g. SEQ. ID NO. 31).

To test if the cell death is due to siRNA-mediated AR silencing, a time-course experiment in the androgen-sensitive LNCaP and the androgen-refractory C4-2 cells was performed. Cells were transfected with the AR siRNAs having SEQ. ID NO. 8 or a scrambled negative siRNA in 5% cFBS. The culture media were changed and the cell number was counted every 3 days.

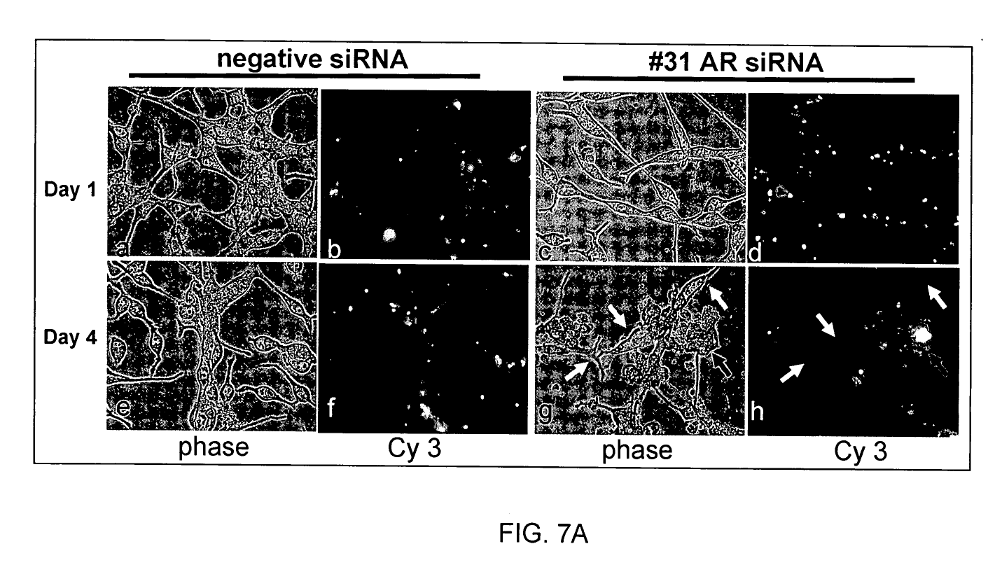

As shown in FIGS. 4A-C, transfection with the AR siRNA resulted in a significant cell death, in which LNCaP cells (FIG. 4A) were more sensitive compared to androgen-refractory C4-2 cells (FIG. 4B). In contrast, the negative control siRNA did not cause cell death. FIG. 4C illustrates that after transfection of LAPC-4 cells with either AR siRNA SEQ. ID NO. 8 or a pooled AR siRNA mixture resulted in massive cell death was observed about four days after siRNA transfection. These data suggest that AR gene silencing mediated by siRNAs in accordance with the present invention leads to cell death regardless of androgen dependency, although the androgen-refractory C4-2 showed a delayed response compared to the androgen-sensitive LNCaP cells.

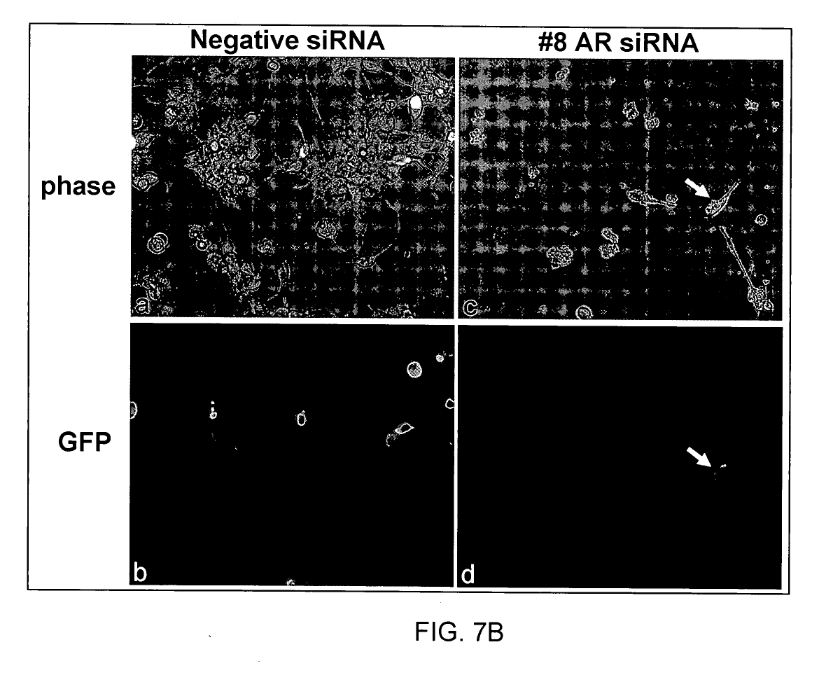

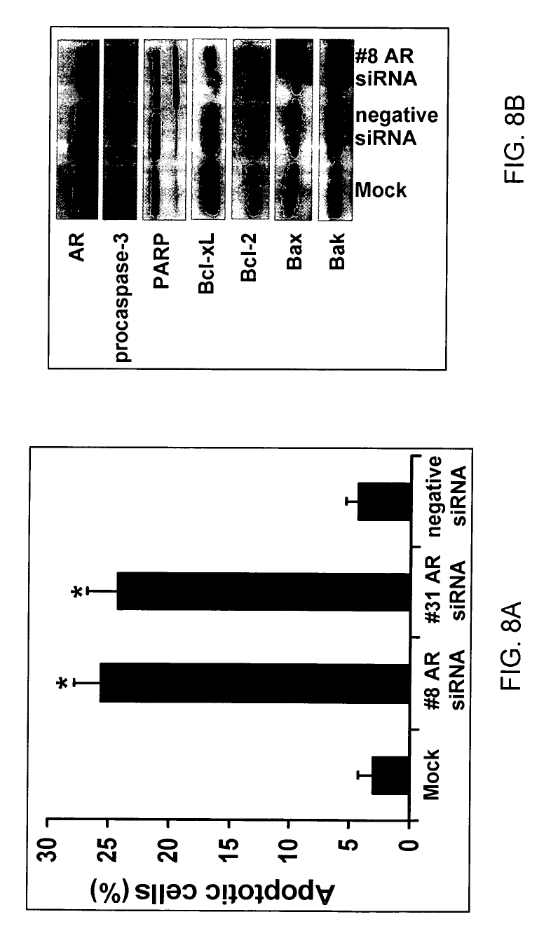

The present invention also investigated if the AR siRNA-induced cell death was simply due to a cellular response to the degraded AR mRNA mediated by the siRNA. The experiments were conducted using an AR null prostate cancer cell PC-3 with or without exogenous human AR expression and the androgen-refractory LNCaP-Rf cells. Briefly, about 10³ cells were plated in 6-well plates with RPMI 1640 plus about 10% charcoal-stripped serum and allowed to attach overnight. The cells were then transfected with 10 nM siRNA having SEQ. ID NO. 8 or SEQ. ID NO. 31 as indicated in FIGS. 5 and 6. Cell growth was monitored daily for seven days with phase contrast optics.

As shown in FIG. 5, transfection of the AR siRNAs reduced the survival rate more than 95% only in the androgen refractory LNCaP-Rf cells, while cell survival was not affected in either PC-3/AR or PC-3/Neo cells. Surprisingly, the siRNA-transfected cells started to die on day four after transfection. On day seven, the survival rate of the siRNA transfected cells reduced in more than about 95% compared to control or mock transfected cells (FIG. 5, FIG. 6 panel f, and FIG. 6 panel g). However, no effect was observed in the siRNA-transfected PC-3/AR or PC-3/Neo cells (FIG. 5). Furthermore, addition of the siRNA alone (FIG. 6 panel b & panel c) or the OLIGOFECTAMINE reagent alone (FIG. 6 panel e) did not affect cell survival. In addition, the GSK-3β siRNA did not show any notable effect on cell survival (FIGS. 6 panel d & 6 panel h). These data suggest that the AR siRNA-induced cell death in the endogenously AR-harboring cells is not a cellular response to siRNA-mediated AR mRNA degradation but due to a disruption of the survival machinery that is dependent on the AR. In contrast, in the AR-null cells, like PC-3/Neo, the survival machinery does not depend on the AR, although an exogenous AR gene is expressed.

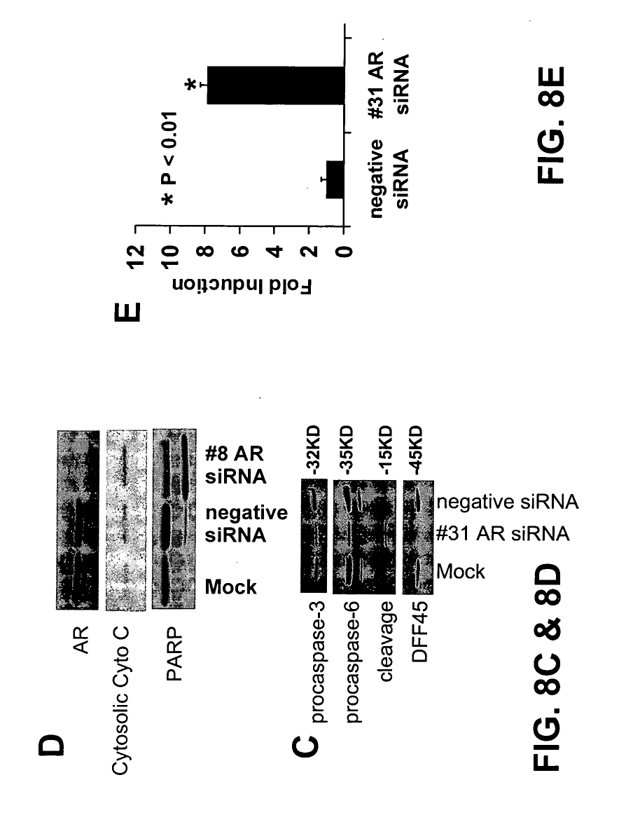

To visualize the specificity of the AR siRNA-induced cell death, the present invention labeled the AR siRNAs with a fluorescent dye (Cy3) and then transfected them into androgen-refractory LNCaP cells. The cells were maintained in about 5% cFBS and cell death was monitored daily. As shown in FIG. 7A, the labeled siRNA was seen inside the cell in a large population of the cells, indicating a successful transfection. Most interestingly, only the dying cells showed a positive labeling, and living cells showed negative labeling (FIG. 7A, panels g and h), indicating the specific effect of the siRNA-induced cell death on the transfected cells.

To further confirm this specificity, a GFP-fused human AR construct (PGFP-hAR) was co-transfected with the siRNAs into LNCaP cells. In this case, it was predicted that the GFP-positive cells (indicating no AR silencing) would be living cells if the AR siRNA-induced cell death is specific. As expected, transfection with the control siRNA did not affect cell survival and GFP-AR expressions (FIG. 7B, panel a&b), but the AR siRNA having SEQ. ID NO. 8 induced significant cell death. Consistent with the first approach, cell death was seen in parallel with GFP-AR knockdown and inhibition while the living cells still maintained GFP-AR expression (FIG. 7B, panel c). These data provide strong evidence that the siRNA-mediated AR silencing specifically leads to cell death in those affected cells.

### EXAMPLE 3

### AR siRNA-Induced Cell Death is Via an Apoptotic Pathway as Evidenced by Caspase Proteolysis, PARP Cleavage, and Release of Cytochrome c

It has been demonstrated that androgen ablation results in apoptotic cell death in prostate epithelium and prostate cancer cells (Kerr 1977, Denmeade 1996). In this example, the present invention investigated whether AR siRNA-induced cell death occurs via an apoptotic pathway.

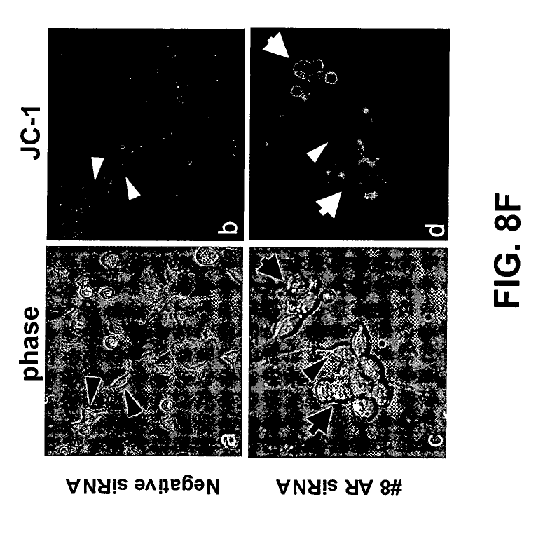

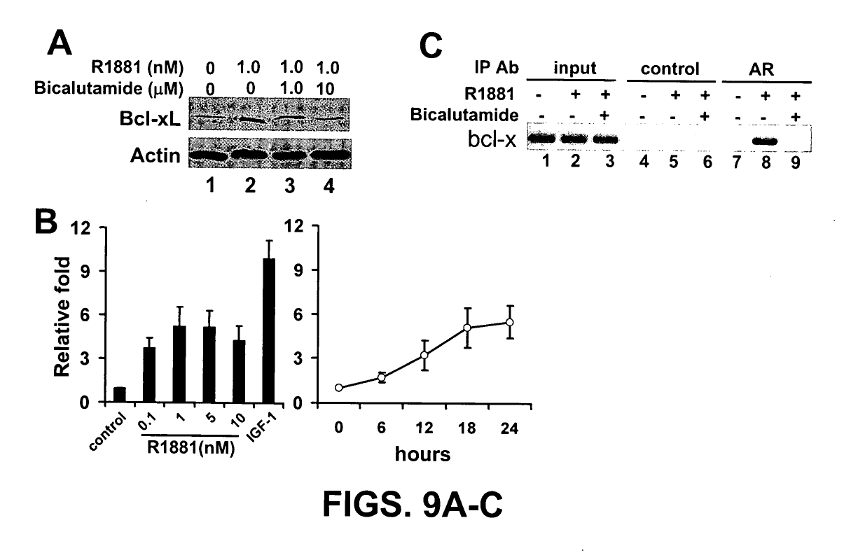

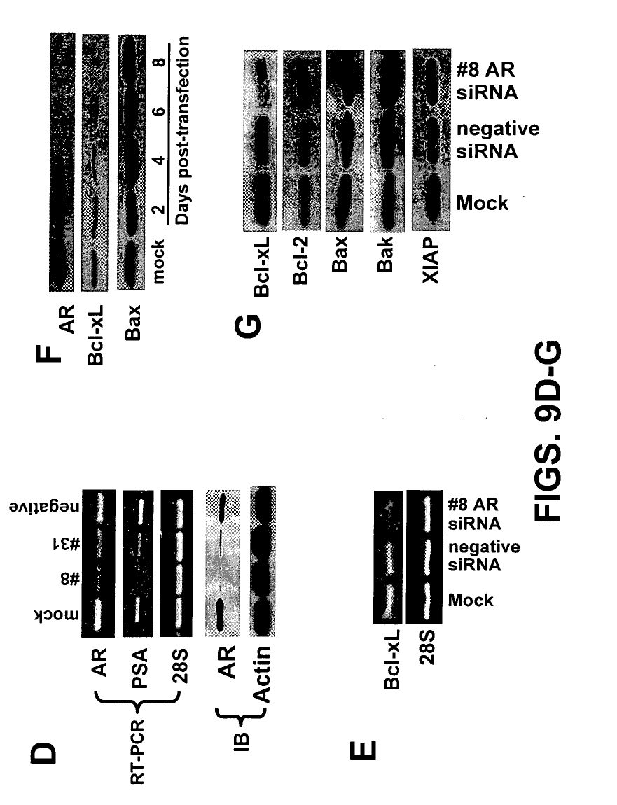

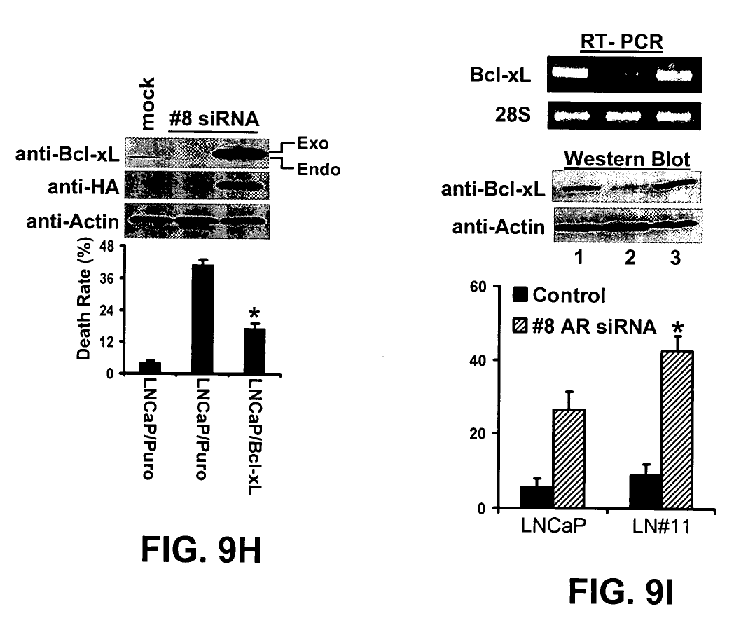

To determine if AR silencing-induced cell death is an apoptotic response, the change of the membrane phospholipid phosphatidylserine (“PS”) which is translocated from the inner to the outer leaflet of the plasma membrane during apoptosis (Martin 1995) was first detected. As shown in FIG. 8A, by measuring the number of FITC-positive cells, it was determined that transfection of the LNCaP cells with the AR siRNAs of the present invention (SEQ. ID NO. 8 and SEQ. ID NO. 31) induced significant apoptotic cell death, while the control siRNA had no effect.

Since apoptotic cell death is associated with caspase proteolysis (activation) and PARP cleavage (Gross 1999), the occurrence of the proteolytic process of two caspases, caspase-3 and Caspase-8, and PARP by western blot was next detected. As shown in FIG. 8B, the AR siRNA having SEQ. ID NO. 8 induced significant reduction of the procaspase-3 (evidence for proteolytic activation) and PARP cleavage, whereas caspase-8 was not processed in the LNCaP cells. Similar results were also seen when LAPC-4 or C4-2 cells were used (data not shown).

Similarly, as shown in FIG. 8C, transfection with the AR siRNA having SEQ. ID NO. 31 into LNCaP cells induced significant reduction of the procaspase-3 and -6, and DFF45 (evidence for proteolytic activation or cleavage). Similar results were also seen when LAPC-4 or C4-2 cells were used (data not shown).

The presence of cytochrome c in the cytosol is a critical event required for the correct assembly of the apoptosome, subsequent activation of the executioner caspases and induction of cell death (Li 2004). To evaluate the release of cytochrome c, the cytosolic fraction of the cellular protein was collected six days after siRNA transfection. As shown in FIG. 8D, when AR siRNA having SEQ. ID NO. 8 was transfected into the cells, cytochrome c was detected in the cytosolic fraction that was in parallel with the AR being inhibited.

Consistently, the catalytic activity of caspase 3 (fold induction) was significant increased when AR siRNA SEQ. ID NO. 31 was used compared to negative control siRNA (FIG. 8E). Thus, these data clearly demonstrated that the mitochondrial apoptotic mechanism is activated by the AR siRNAs.

Since loss of the mitochondrial transmembrane potential (Δ_{ψm}) is considered to be one of the central events in apoptotic death that leads to incapacitation of the mitochondria, release of cytochrome c, and activation of the caspase pathway, the integrity of mitochondrial membrane using the fluorescent dye JC-1 was also tested (Petit 1995). Upon entering the mitochondrial negative transmembrane potential in healthy cells, JC-1 forms red fluorescent aggregates. When the transmembrane potential is low, as in many cells undergoing apoptosis, JC-1 exists as a monomer and produces green fluorescence. Consistent with this notion, green fluorescence was observed in dying cells after transfected with AR siRNA SEQ. ID NO. 8 (as pointed out by arrows in FIG. 8F) while living cells remained normal membrane potential (red fluorescence as pointed with arrow-head in FIG. 8F).

### EXAMPLE 4

### AR siRNA-Induced Cell Death Via an Apoptotic Pathway Involving Bcl-x_{L}

1. Androgen Regulates Bcl-x_{L} Expression at a Transcriptional Level.

As discussed above, it has been widely accepted that prostate growth and differentiation is androgen-dependent, and the AR plays a critical role in the development and progression of prostate cancer. Androgen withdrawal triggers apoptosis in both normal and malignant prostate epithelial cells but hormone-refractory prostate cancer cells do not undergo apoptosis, suggesting that AR-mediated survival signal is reactivated or prostate cancer cells may utilize alternative cellular pathways for their survival. So far, however, little is known about the mechanism for AR-mediated survival.

In a large-scaled genome-wide gene expression analysis (Holzbeierlein 2004), it was noticed that Bcl-x_{L}, the anti-apoptotic member of the Bcl-2 family, was significantly down-regulated after androgen ablation therapy, while Bcl-x_{L} expression was dramatically increased in late stage of the disease including hormone-refractory tumors compared to the primary and hormone-treated tumors. In contrast, other two major members, Bcl-2 and Bax, of the family showed no significant alteration during androgen ablation therapy or progression. These data suggest that expression of bcl-x gene might be regulated by androgens.

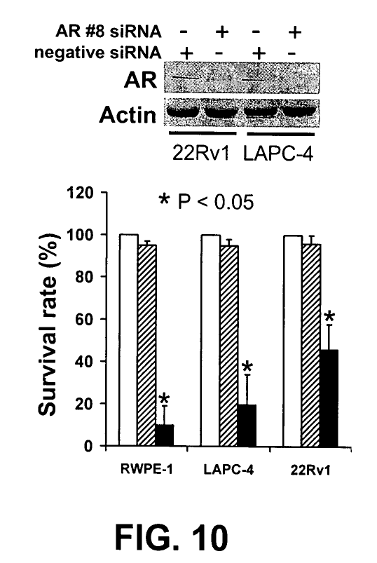

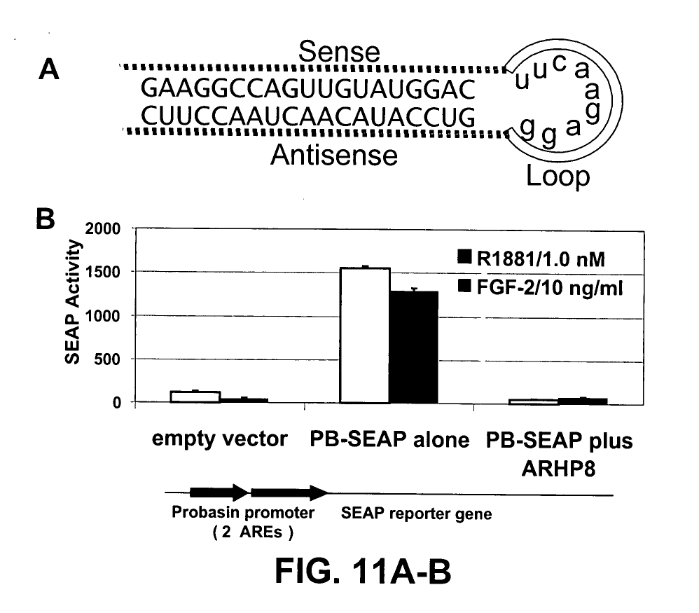

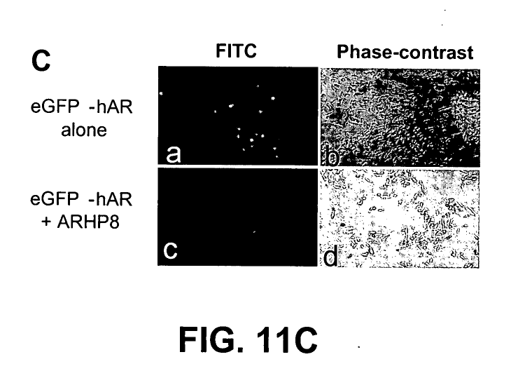

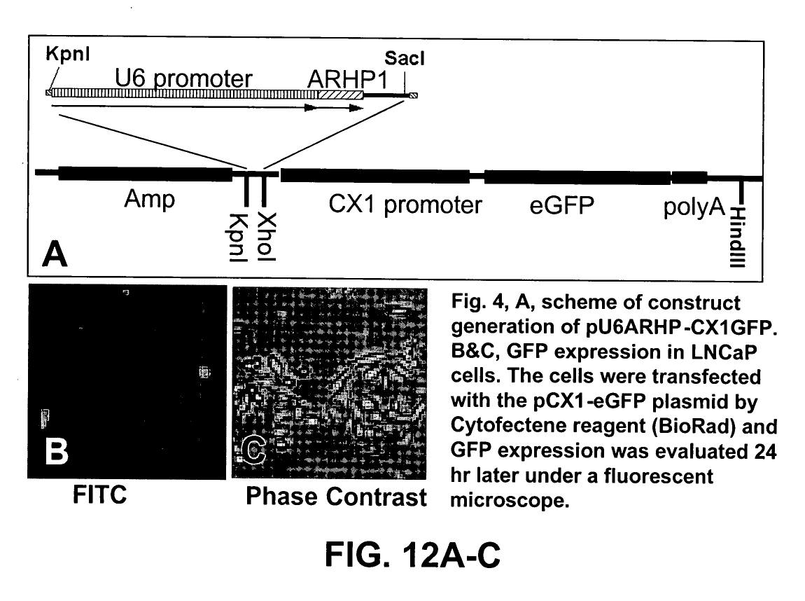

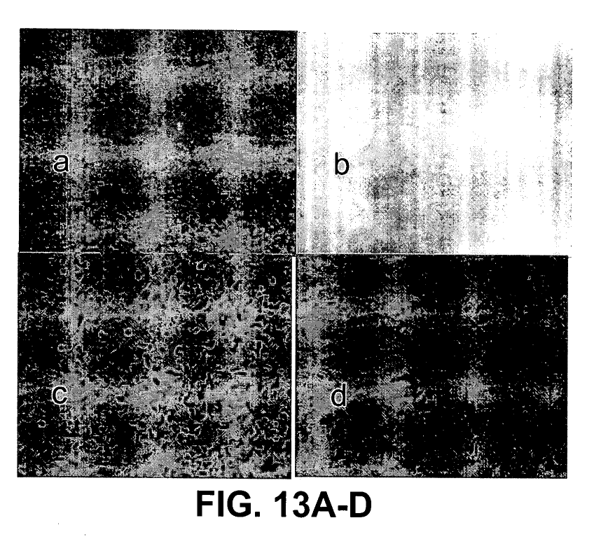

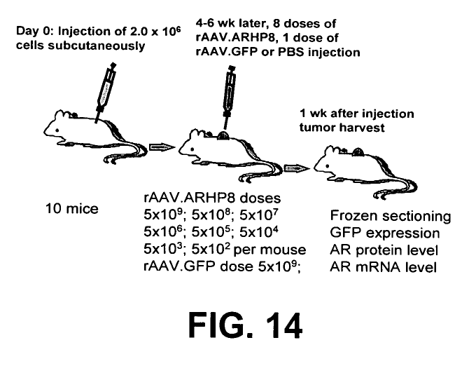

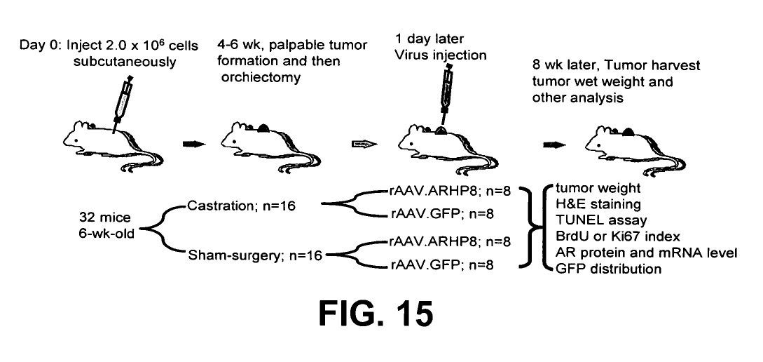

To shed light onto the significance underlying the response of Bcl-x_{L} reduction to androgen ablation therapy in prostate cancers, the AR involvement in the transcriptional regulation of Bcl-x_{L} gene was investigated. First, human prostate cancer LNCaP cells that harbor an endogenous mutant AR gene were treated with a synthetic androgen R1881 in the presence or absence of antiandrogen bicalutamide. Western blot analysis showed that R1881 treatment induced a significant increase of Bcl-x_{L} protein expression that was blocked by a pretreatment of bicalutamide (FIG. 9A). Next, a luciferase reporter gene assay was utilized to test if androgen stimulates the promoter activity of the bcl-x gene. As shown in FIG. 9B, R1881 strongly stimulated the Bcl-x_{L} promoter activity in a dose-dependent and time-dependent manner, as did the IGF-1, which was reported to stimulate Bcl-xL expression and to play a role in androgen-independent progression of prostate cancer (Parrizas 1997, Nickerson 2001).

By analyzing the bcl-x promoter sequence (GenBank accession number D30746), three potential androgen responsive element-like (“ARE-like”) motifs were noticed, SEQ. ID NO. 46: -463/-446, 5′-tgtgatacaaaagatct-3′; SEQ. ID NO. 47: -588/-577, 5′-tgtcgccttct-3′; SEQ. ID NO. 48: -613/-605, 5′-tggttcct-3′, as suggested by previous reports (Devos 1997, Claessens 2001). To determine if the AR binds to this region of the promoter in the bcl-x gene, a protein-DNA interaction assay (ChIP assay) was performed. As shown in FIG. 9C, the R1881 treatment greatly induced the AR binding to the promoter region (-600/-390) of the bcl-x gene, while pretreatment with bicalutamide abolished this interaction. These data clearly demonstrated that the AR is involved in transcriptional regulation of the bcl-x gene in prostate cancer.

2. siRNA-Mediated AR Silencing Results in Down-Regulation of the bcl-x Gene Expression.

To further demonstrate the role of androgen (and the AR) in regulation of bcl-x gene expression, the AR protein was knocked down using siRNAs having SEQ. ID NO. 8 and SEQ. ID NO. 31. It will also be appreciated that a well-established androgen target prostatic specific antigen (“PSA”) was also down-regulated (FIG. 9D). In parallel, the AR protein level was also decreased as assessed by a Western blot. This knocking-down effect was achieved as a sequence-specific event since a negative control siRNA with scrambled sequence had no effect on AR protein or PSA mRNA levels (FIG. 9D). These results demonstrate that the RNAi machinery is functional in prostate cancer cells.

In view of androgen stimulation of the bcl-x gene expression, an investigation as to whether AR silencing results in down-regulation of Bcl-x_{L} expression was implemented. Transfection of the siRNA having SEQ. ID NO. 8 induced a dramatic decrease of the Bcl-x_{L} mRNA as shown in FIG. 9E. To better illustrate the relationship of Bcl-x_{L} reduction with AR silencing, a time-course experiment was conducted and found that Bcl-x_{L} expression was gradually decreased in parallel with the AR level (FIG. 9F), while Bcl-2, Bax, Bak and XIAP proteins remained unchanged (FIGS. 9F and 9G). These data further confirmed the role of the AR in regulation of the bcl-x gene expression.

3. AR siRNA-Induced Apoptosis was Partially Inhibited by Ectopic BCl-x_{L} Expression

In view of the anti-apoptotic effect of Bcl-x_{L} protein, it was hypothesized that the AR promotes cellular survival by up-regulating the bcl-x gene expression through a transcriptional mechanism in prostate cancer cells. Therefore, Bcl-x_{L} expression will decrease if the AR is knocked down, which subsequently results in apoptosis due to an imbalance between the pro- and anti-apoptotic members of the Bcl-2 family. Thus, it was hypothesized that an enforced Bcl-x_{L} expression will protect cell from apoptosis while AR is silenced. To assess the protection effect of Bcl-x_{L}, a stable LNCaP subline over-expressing human Bcl-x_{L} protein controlled by a CMV promoter (LNCaP/Bcl-x_{L}) or a control subline with an empty vector (LNCaP/Puro) were established. Consistent with the results obtained from the parental cells (FIG. 9F), exposure of those LNCaP subline cells to AR siRNA having SEQ. ID NO. 8 resulted in a decrease of endogenous but not exogenous Bcl-x_{L} protein (FIG. 9H). Most significantly, enforced Bcl-x_{L} expression partially inhibited cell death induced by AR siRNA transfection in LNCaP/Bcl-x_{L} cells compared to the controls.

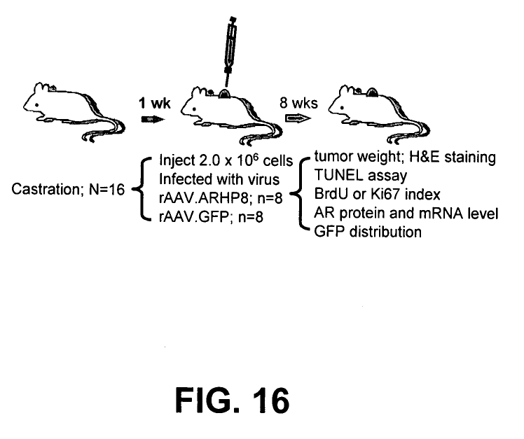

These data demonstrated that Bcl-x_{L} is involved in AR-mediated survival of prostate cancer, and the reduction of Bcl-x_{L} expression after AR silencing represents a mechanism for the AR siRNA-induced apoptosis. In addition, while establishing a subclone for stable BCl-x_{L} expression in LNCaP cells, a clone (LN#11) was unexpectedly obtained, in which the expression of BCl-x_{L} expression was dramatically reduced for unknown reasons, as confirmed by RT-PCR and Western blot (FIG. 91, upper panel). By taking the advantage of this subclone of LNCaP cell line, the involvement of Bcl-x_{L} in AR-mediated survival was confirmed (FIG. 91, lower panel). Reduction of Bcl-x_{L} expression led to a significant increase in AR siRNA-mediated cell death compared to the parental LNCaP cells, although loss of Bcl-x_{L} alone did not cause profound cell death, indicating that multiple downstream factors, except Bcl-x_{L}, are mediating AR survival signal.

### EXAMPLE 5

### Specificity for Protstate Cancer

In addition to those commonly used prostate cancer cells as mentioned above, the cell death response to the AR siRNA in three more prostate epithelial cell lines (LAPC-4, RWPE-1 and 22Rv1) and two breast cancer cell lines (MCF-7 and T47D) was tested to verify the specificity of AR siRNA-induced cell death. The RWPE-1 is a non-tumorigenic prostate epithelial cell line (Bello 1997) while the 22Rv1 is a hormone-refractory prostate cancer cell derived from CWR22 xenograft (Bello 1997). Although the 22Rv1 cells, like C4-2 cells, showed a delayed response to AR siRNA-induced cell death, the non-tumorigenic RWPE-1 cell demonstrated a rapid death response even faster than LAPC-4 and 22Rv1 cells (FIG. 10). The selected data for AR siRNA-induced AR protein knockdown in 22Rv1 and LAPC-4 cells is shown in FIG. 10. However, the two breast cell lines did not show any cell death response to AR siRNA (data not shown), indicating a tissue-specific survival mechanism under the control of the AR.

### EXAMPLE 4

### Plasmid Construction Bearing a siRNA Hairpin for AR Gene Silencing

In order to maintain sustained gene silencing in cells, a common approach is to stably transfect the cells with a hairpin-structured siRNA under the control of a promoter, such as CMV, U6 or H1 RNA polymerase promoter. As exemplary hairpin structure based on the siRNA having SEQ. ID NO. 8 is shown in FIG. 11A. It will be appreciated that similar hairpin structures may be developed for any of the siRNA sequences of the present invention.

The oligonucleotides were synthesized by Integrated DNA Technologies, Inc. (“IDT”) and subcloned into the ApaI-EcoRI sites of the pSILENCER 1.0-U6 vector according to the manufacturer's instruction (Ambion, Inc.). The sequence of the resulted plasmid (termed as pU6-ARHP8) was verified by direct sequencing and its effect on AR gene silencing was determined by two different assays described as follows.

First, the pU6-ARHP8 construct was co-transfected with an AR responsive reporter Probasin-SEAP (Xie 2001) (obtained from Dr. David Spencer, Baylor College of Medicine, Houston, Tex.) into LNCaP cells and SEAP activity was measured 24 hours later after addition of synthetic androgen R1881 or fibroblast growth factor 2 (“FGF-2”), which can induce AR transactivation independent of androgen (Culig 1994). As shown in FIG. 11B, pU6-ARHP8 transfection resulted in a complete blockage of androgen-stimulated or FGF2-stimulated AR responsive gene expression.

In this example, the pU6-ARHP8 was co-transfected with a plasmid construct bearing GFP and human AR fusion protein (Ozanne 2000) (peGFP-hAR, obtained from Dr. Craig Robson, Newcastle University, UK) into LNCaP cells and monitored eGFP-hAR expression at the protein level under fluorescence microscope. As shown in FIG. 11C, eGFP-hAR expression was dramatically eliminated when the cells were co-transfected with pU6-ARHP8 and peGFP-hAR. Reduced cellular proliferation was also observed in the co-transfected cells (FIG. 11C, panel d) comparing to the peGFP-hAR transfection control (FIG. 11C, panel b), indicating that knocking down the AR protein in prostate cancer cells leads to reduced cellular proliferation, which is consistent with a previous report (Zegarra-Moro 2002). This example thus demonstrates that the siRNA hairpin mediated effective AR gene silencing in human prostate cancer cells.

In order to visibly monitor the transfection efficiency of the AR hairpin in cells, the U6-ARHP8 expressing cassette (451 bp, KpnI-SacI fragment from the pU6-ARHP8 construct) were subcloned into the KpnI-XhoI sites on pCX1-eGFP vector (obtained from Dr. Jie Du, Department of Medicine, University of Texas Medical Branch), as outlined in FIG. 12A. The SalI and XhoI ends were blunted. The CX1 promoter is a hybrid promoter composed of the CMV immediate early enhancer and a chicken β-globin promoter, and it has been shown to drive high levels of eGFP expression in a wide variety of tissues in transgenic mice (Okabe 1997). The CX1 promoter-driven eGFP expression was tested in prostate cancer LNCaP cells as shown in FIGS. 12B and 12C. The resulted plasmid construct, termed as pU6ARHP8-CX1GFP, will give off fluorescent light when it is transfected into cells. The KpnI-HindIII fragment (3554 bp) containing the two expressing cassettes (U6-ARHP8 and CX1-eGFP) will be released for adeno-associate virus construction.

D. Infection of Prostate Cancer Cells with Type-2 AAV

Since knocking down of the AR gene expression will result in growth inhibition in prostate cancer cells, the present invention preferably utilizes a viral vector approach for infection. Among the viral vehicles for gene delivery purpose, adeno-associated virus type 2 (“AAV-2”) is a non-pathogenic human parvovirus that is being developed as a gene therapy vector for the treatment of numerous diseases. The major advantage of wild-type AAV-2 is its ability to preferentially integrate its DNA into a 4-kilobase region of human chromosome 19, designated AAVS1 (Kotin 1992), which is highly desirable in a gene therapy vector. Thus, the AR siRNA hairpin is preferably expressed constantly in the cells, thereby avoiding the drug-selection procedure. Further, AAV DNA has been found in human semen and testis tissue, suggesting the permission of viral transduction for the prostate-derived cells.

To further confirm that prostate cancer cells are infectable by a recombinant type-2 AAV (“rAAV2”), the present invention tested two commonly used prostate cancer cell lines, LNCaP and PC-3. As shown in FIG. 13, both cell lines showed convincing efficiency of permissive infection with the rAAV2 (1.0×10⁴ viral partials per cell) carrying different reporter genes: alkaline phosphatase (AP, FIGS. 13 A&B, in which positive result reads as dark-blue dots on top of the pink background while negative one reads nothing), and lacZ (FIGS. 13 C&D, in which the green dot represents positive).

### PROPHETIC EXAMPLE 1

This example involves the generation of a recombinant AAV for long-term expression of a hairpin-structured AR siRNA in vivo.

A. Rationale and Strategy:

As discussed above, AAV is a non-pathogenic and single strand Parvoviridae family DNA virus. Recombinant AAV (“rAAV”) has been used extensively as gene delivery vehicles to transduce a wide range of cells in vitro and in vivo (Berns 1996, Kessler 1996, Xiao 1996). In rAAV, all the wild-type AAV open reading frames (“ORFs”) are replaced by the customer-favored transgene expression cassette. Recombinant AAV are capable of transducing a broad range of cell types and transduction is not dependent on active host cell division. High titers, typically greater than 10⁸ viral partical/ml, are easily obtained in the supernatant and ≧10¹¹-10¹² viral partical/ml with further concentration. The gene of interest is either persisted as episomal DNA or integrated into the host genome so expression is long term and stable. The rAAV viral stocks are produced with a cis-plasmid in which the transgene expression cassette is flanked by viral inverted terminal repeats (“ITRs”). All the other factors that are required for rAAV replication and packaging are provided in trans by helper plasmids, viruses and/or producer cell lines (Owens 2002).

B. Experimental Design and Methods:

1a. Generation of a Recombinant AAV for the AR siRNA Hairpin Expression

This example will use the type-2 AAV ITR from pSub2Ol (Samulski 1987) for all the recombinant AAVs, and package them with a type-2 capsid. The rAAV will be generated as previously described (Duan 1997; Duan 1998; Duan 2002). To generate pAAV.ARHP8, carrying the U6-ARHP8 expression cassette for the AR siRNA (preferably having SEQ. ID NO. 8 or 31) hairpin plus the CX1-GFP expression cassette for GFP, the 3554 bp fragment will be released from the pU6ARHP8-CX1GFP construct, by KpnI-HindIII digestion and blunted into the XbalI sites in pSub201. The pAAV.GFP, carrying the CX1-GFP expression cassette only (3103 bp), will also be released by KpnI-HindIII digestion from the pCX1-GFP (obtained from Dr. Jie Du, University of Texas Medical Branch, Galveston, Tex.) and blunted into the XbalI sites in pSub201. The intactness of the inverted terminal repeat sequence in all the clones will be screened by three restriction enzymes including BssHII, MscI and SmaI as described previously (Duan 2002). The correct clones will be further confirmed by direct sequencing. The recombinant viral stocks will be generated with an adenovirus-free transient transfection system as previously described (Duan 2002). The viral fractions will be pooled and dialyzed in HEPES-buffered saline (20 mM HEPES, 150 mM NaCl, pH 7.8). Viral aliquots will be stored at −80° C. in 5% glycerol until use. The viral titers will be determined by quantitative slot blots using cis plasmid standards. The average yield is expected to be about 5×10¹² viral particles/ml. The contamination of wild-type AAV-2 will be determined, which is expected to be one functional particle per 1×10¹⁰ rAAV particles (Duan 2002).

1b. Evaluate the Efficiency of the Resultant rAA V.ARHP8 for AR Gene Silencing

To evaluate the effect of the resultant rAAV carrying the AR siRNA hairpin expression cassette (rAAV.ARHP8) on AR gene silencing, this example will monitor AR expression at the protein level by Western blot (anti-AR antibody clone 441, Santa Cruz Biotech Inc.) and AR responsive reporter (Probasin-SEAP) gene assay, as well as at the RNA level (RT-PCR) described in detail as follows.

The present invention will test two prostate cancer cell lines, including LAPC-4 (obtained from Dr. Charles Sawyers, UCLA, Calf.) that has a wild type AR (Klein 1997), and LNCaP that harbor a mutant AR (Van Steenbrugge 1991). The infection efficiency will be monitored under a fluorescent microscope since the resultant rAAV carries a GFP expression cassette. The optimal viral infection condition for different cell lines will be determined. After 3-5 days, the cells will be harvested for AR protein assessment.

In a separate experiment, a functional assay will be conducted by using an androgen receptor responsive promoter reporter (Probasin-SEAP) as described previously (Tosetti 2003; Li 1997).

In addition, the present invention will investigate the AR transcript (mRNA level) in the infected cells by RT-PCR with primers of sense, SEQ. ID NO. 49: 5′-AGATGGGCT TGACTTTCCCAGAAAG-3′and antisense SEQ. ID NO. 50: 5′-ATGGCTGTCATTCA GTACTCCTGGA-3′). GAPDH primer in another tube will serve as an internal control for the RT-PCR reaction as described previously (Tsuka 1998). The control virus rAAV.GFP will serve as a negative control.

1c. Expected Result

By adjusting the doses and duration of the rAAV infection, this example will knock down the AR expression effectively in the cells. The present invention will compare the efficiency of the recombinant rAAV.U6ARHP8 on AR gene silencing with purified siRNA oligonucleotides or the plasmid construct pU6-ARHP8, in which their efficiencies were confirmed previously. By completing the experiments, the present invention will obtain the viral stock of rAAV.U6ARHP8 and rAAV.GFP for future experiments.

### PROPHETIC EXAMPLE 2

This example involves determining of the essential need of the AR for androgen-independent growth of prostate cancer.

Rationale and Strategy:

As mentioned above, ligand-independent activation of the AR is one of the proposed mechanisms for androgen-independent progression of prostate cancer. Disruption of the AR signaling suppresses cell proliferation of prostate cancer cells regardless of androgen responsiveness in vitro (Zegarra-Moro 2002).

The present invention found that transfection of the AR siRNA reduced cell survival in LNCaP-Rf. To further address this issue in vivo, the present invention will determine if the resultant rAAV.ARHP8 (from Prophetic Example 1), which triggers AR gene silencing in cells, can inhibit tumor growth of prostate cancer xenograft established from prostate cancer cells or human prostate cancer tissue.

### PROPHETIC EXAMPLE 2A

This example involves a pilot experiment for evaluation of rAAV.ARHP8-mediated AR gene silencing in prostate cancer xenograft of mouse model

First, the present invention will use PC-3/AR cells (which have higher tumor formation rate) to optimize experimental condition and evaluate the efficiency of rAAV.ARHP8-mediated AR gene silencing in vivo. FIG. 14 shows briefly the experimental design.

Ten animals will be used. A total of about 2.0×10⁶ viable cells, as determined by trypan blue exclusion, will be resuspended in RPMI-1640/10% fetal bovine serum mixed with a 4:1 v/v ratio of MATRIGEL (Catalog#356237, BD Bioscience) vs cells. The cells will be injected subcutaneously (27-gauge needle, 1-ml disposable syringe, and total volume 0.1 ml/site at two sites per mouse) into the rear flank of six-week old castrated athymic male mice (Balb/c, Charles River Laboratories). When the tumor is palpable (i.e. 50-100 mm³ in 4-6 weeks), 8 different doses (log-dilution, 5×10²-5×10⁹ viral particles/10 μl total volume) of the recombinant rAAV.ARHP8 obtained from Prophetic Example 1 will be injected into the tumor (one dose per tumor). In addition, one animal will receive either control virus rAAV.GFP (maximum dose of 5×10⁹ viral particles in a volume of 10 μl) or 10 μl PBS solution as negative control. The tumor will be harvested one week later, and half of the tumor will be snap-frozen in liquid nitrogen with OCT embedding medium. After sectioning the frozen tissue (5 μM in thickness), the viral infection efficiency will be evaluated under fluorescent microscopy for GFP expression. The other half of the tumor specimen will be snap-frozen in liquid nitrogen and then stored in −80° C. for AR protein and total RNA analysis. The AR protein and mRNA expression will be assessed by Western blot and RT-PCR, respectively as described. The optimal dose for highest infection rate and AR gene silencing efficiency will be defined.

### PROPHETIC EXAMPLE 2B

This example evaluates the effect of rAAV.ARHP8-mediated AR gene silencing on acquisition of the androgen-independent phenotype by prostate cancer LNCaP and LAPC-4 cells in vivo.

For this prophetic example, the present invention will use two androgen-dependent prostate cancer cell lines, LNCaP and LAPC-4, which maintain the androgen-dependent phenotype. Once inoculated in nude mouse subcutaneously, the cells can form tumors. The tumor growth will be arrested after castration for a period (around 4 weeks) and then it will re-grow in an androgen-independent manner (Klein 1997, Van Steenbrugge 1991, Horoszewicz 1983). Therefore, this feature makes them as suitable model for accessing the effect of various factors on androgen-independent transition.

FIG. 15 shows briefly the experimental design. For each of the cell lines, LNCaP or LAPC-4, thirty-two six-week old athymic male mice (Balb/c, Charles River Laboratories) will be used. The exponentially growing LNCaP or LAPC-4 cells in culture will be trypsinized, neutralized with the culture medium containing 10% FBS, and washed once in the same medium. A total of about 2.0×10⁶ viable cells, as determined by trypan blue exclusion, will be resuspended in RPMI-1640/10% FBS mixed with a 4:1 v/v ratio of MATRIGEL (Catalog#356237, BD Bioscience) vs cells (27-gauge needle, 1-ml disposable syringe, total volume 0.1 ml/site at 2 sites per mouse) and then injected into the rear flank of the animals. Tumor development will be followed in individual animals. When the tumor becomes palpable (i.e., 50-100 mm³ in 4-6 weeks), the animals will be randomly assigned into two groups (16 mice per group). One group of animals will receive a surgical castration (bilateral orchiectomy) while the other group receives a sham-operation only. One day later, half of the mice (8 mice for each subgroup) from each group will receive an intratumoral injection of the optimized dose (determined in the pilot experiment as described in the previous section) of either the rAAV.ARHP8 or rAAV.GFP virus stock. The tumor growth will be followed for another eight weeks by sequential caliper measurements of length, width, and depth every week and any androgen-independent tumor growth will be recorded for each subgroup. The serum level of the human AR target gene product prostate specific antigen (HPSA) has been used to monitor tumor growth in nude mice (Csapo 1988). The present invention will also measure the serum level of hPSA in mouse blood to determine the efficiency of androgen. receptor silencing in response to the rAAV injection. Mouse blood samples will be obtained from tail incision and the hPSA level will be measured every week by the Tandem-R assay (Hybritech Corp, San Diego). On the last day of the experiment, one hour before sacrifice, the animals will be injected intraperitoneally (i.p.) with 0.5 ml of a 10-mM solution of BrdU from an in situ proliferation assay kit (Roche Diagnostics, Indianapolis, Ind.) as recommended by the manufacturer. Immunohistochemistry for proliferating markers including BrdU and Ki-67 (monoclonal antibody Cat#F0722, DakoUSA) and AR protein expression will be conducted by the procedure as described previously (Li 1998, Dou 1999). The present invention will also measure apoptosis by means of terminal deoxynucleotidyl transferase-mediated dUTP nick and labeling (“TUNEL”) analysis (APOALERT® DNA fragmentation assay kit, Cat#K2024-1, Clontech) in the tumor samples. Tumor volume, incidence and proliferating index (BrdU labeling and KI-67 staining) and apoptotic data will be analyzed statistically (StartWork software; BrainPower). The level of significance will be set at p value<0.05.

### PROPHETIC EXAMPLE 2C

This example will evaluate the effect of rAAV.ARHP8-mediated AR gene silencing on tumor growth of prostate cancer xenograft established from androgen-independent cell lines.

FIG. 16 shows briefly the experimental design of this prophetic example. The present invention will use two androgen-independent prostate cancer cell lines, LNCaP-C4-2 (Wu 1994, Thalmann 1994) (obtained from UroCor, Oklahoma, Okla.) and LNCaP-LNO (Soto 1995) (obtained from W. M. van Weerden, PhD, Erasmus University Rotterdam, Holland), which form tumor and grow rapidly even in castrated nude mouse when inoculated subcutaneously. For each cell line, sixteen six-week old athymic male mice (Balb/c, Charles River Laboratories) will be used and surgically castrated (bilateral orchiectomy) before tumor cell implantation. The cells exponentially growing LNCaP-LNO or LNCaP-C4-2 will be infected ex vivo for 24 hours before inoculation into animal with the rAAV.ARHP8 or rAAV.GFP. The optimized dose for highest infection rate (determined in Prophetic Example 1) will be used. One week after castration, the animals will be randomly assigned to two experimental groups (rAAV.ARHP8 group and rAAV.GFP group) of eight animals each, and will be injected with a total of about 1.0×10⁶ viral viable tumor cells, as determined by trypan blue exclusion. Before injection, the cells will be resuspended in RPMI-1640/10% FBS mixed with a 4:1 v/v ratio of MATRIGEL (Catalog#356237, BD Bioscience) vs cells and then injected (27-gauge needle, 1-ml disposable syringe, total volume 0.1 ml/site at two sites per mouse) into the rear flank of the animals. The tumor growth will be followed in individual animals by sequential caliper measurements of length, width and depth for eight weeks. Any androgen-independent tumor growth will be recorded for each group. Mouse blood sample will be obtained from tail incision and the serum level of hPSA will be measured every week by the Tandem-R assay (Hybritech Corp, San Diego). On the last day of the experiment, one hour before sacrifice, the animals will be injected intraperitoneally (i.p.) with 0.5 ml of a 10-mM solution of BrdU from an in situ proliferation assay kit (Roche Diagnostics, Indianapolis, Ind.) as recommended by the manufacturer. Tumor size and wet weight will be measured. Metastatic tumors (if any) from distant organs or lymph nodes will be harvested. Blood samples will be collected by heart puncture and the serum will be stored at about −80° C. for further analysis. Half of the tumor specimen will be snap-frozen in liquid nitrogen and stored in about −80° C. for AR protein and mRNA analysis. The other half of the tumor specimen will be fixed in about 4% paraformadehyde and 5-micron paraffin-embedded tumor sections will be cut. Tumor sections will be stained with hematoxylin and eosin to determine tumor structure and cellular differentiation, and the extent of tumor necrosis or apoptosis as well. Immunohistochemistry for proliferating markers including BrdU and Ki-67 (monoclonal antibody Cat#F0722, Dako USA) and AR protein expression will be conducted by the procedure as described in our previous publication (Li 1998, Dou 1999) The present invention will also measure apoptosis by means of TUNEL analysis (APOALERT DNA fragmentation assay kit, Cat#K2024- 1, Clontech) in the tumor samples. Tumor volume, incidence, proliferating index (BrdU labeling and KI-67 staining) and TUNEL data will be analyzed statistically (StartWork software; Brain- Power). The level of significance will be set at p value<0.05.

Expected Results and Alternative Approach:

Based on literature (Xiao 1998, Raffo 1995, Passaniti 1992, El Etreby 2000, Gleave 1998), it is anticipated that injection of about 2.0×10⁶ cells or more will lead to tumor formation in the majority of the intact animals for all cell lines. Castration will have no effect on tumor formation for the androgen-independent cells (LNCaP-LNO and LNCaP-C4-2) infected with the control virus. MATRIGEL is a solubilized basement membrane matrix, and is suited for LNCaP or LAPC-4 cells to form tumor in nude mouse. Although the use of MATRIGEL, the tumor formation incidence still varies from 60-80%. Thus, the present example intends to use eight mice per subgroup, about 25-50% more than animals that are needed. To further economize on mice required, the present invention will inject mice in two flanks. In animals bearing two tumors, only one tumor will receive virus and the other one will serve as internal control. The present invention anticipates that intratumoral injection of the rAAV.ARHP8 virus will suppress tumor growth of the LNCaP and LAPC-4 xenografts after castration. Tumor formation rate and tumor size will be significantly reduced in those mice bearing the rAAV.ARHP8-infected LNCaP-C4-2 or LNCaP-LNO xenografts comparing to control infection with rAAV.GFP. In parallel, serum PSA level will be dramatically lower in the rAAV.ARHP8 infection group comparing to the rAAV.GFP group. If a suppressed tumor growth is observed in these two experiments, it would support the hypothesis that the androgen receptor is essential for prostate cancer progression. Next, the present invention will examine the proliferation-related markers, such as BrdU/Ki67 labeling index, and other cell cycle related protein, such as p27^{kip1}, p21^{cip1/Waf1}, cyclin-dependent kinases and cyclin D1, etc.

If there is not a significant difference on tumor growth or tumor formation/growth rate between the two groups of rAAV infection (rAAV.ARHP8 vs rAAV.GFP), although the possibility is extremely low, this example will first investigate the expression level of the AR gene in the tumor tissue by immunohistochemistry (AR protein expression) and RT-PCR (AR mRNA level) methods. Unsuccessful virus infection, for example, limited virus distribution following intratumoral injection is a possible reason. To solve this problem, the present invention will use a recent developed method based on GEL-FOAM (Pharmacia and Upjohn Inc., Kalamazoo, Mich.) or other slow release materials to increase virus distribution. The present invention may adjust the doses of the rAAV to optimize the infection rate, or concentrate the GFP expressing cells (rAAV-infected) by Fluorescent Activated Cell Sorting (“FACS”) before injecting them into the mice. In addition, the present invention may choose another serotype AAV for intratumoral delivery of the AR siRNA hairpin because different serotype of AAV uses different cell surface receptor and probably possesses higher transduction efficiency in prostate cancer xenograft. Finally, the present invention may try another siRNA ( e.g., the AR siRNA having SEQ. ID NO. 31) to trigger AR gene silencing in vivo because different siRNA sequence may have different efficiency once it is expressed in vivo. If a decreased AR protein expression in the tumor specimens while no tumor growth reduction and serum PSA decline is observed, it would suggest that AR is not essential for androgen-independent progression of prostate cancer in vivo. If this is the case, the present invention will investigate if other events, for example, nuclear factor kappa B (“NF-κB”)-related pathways (Chen 2002) or aberrant expression of Bcl-2 family proteins (McDonnell 1992), are involved in androgen-independent progression. Increased anti-apoptotic response or altered intracellular signaling pathways, which are independent of AR transactivation, may participate in androgen-independent progression of prostate cancer.

Once it is observed that AR gene silencing mediated by RNAi mechanism leads to disruption of androgen-independent progression of prostate cancer in vivo, the present invention will proceed on to use a human prostate cancer tissue-derived xenograft in nude mice to test if the recombinant AAV.ARHP8 can eliminate tumor growth. It will also be appreciated that the in addition to treatment for prostate cancer, the siRNAs of the present invention have many other applications, including target validation (for developing novel AR inhibitors), and genomic discovery applications(AR-related biological function) associated with prostate cancer.

It will also be appreciated that the in addition to treatment for prostate cancer, the siRNAs of the present invention have many other applications, including target validation (for developing novel AR inhibitors), and genomic discovery applications (AR-related biological function) associated with prostate cancer.

It will also be appreciated that the delivery route of the siRNA for therapeutic purpose can be achieved in any suitable way, in addition to the rAAV approach using hairpin-structure fragment. Such methods include, but are not limited to, liposome-based systemic approach, and hydrodynamic delivery of naked DNA (bearing the hairpin structure) or pure synthetic siRNA. Such techniques are described in Song Y K, Liu F, Zhang G, Liu D, Hydrodynamics-based transfection: simple and efficient method for introducing and expressing transgenes in animals by intravenous injection of DNA, Methods Enzymol. 2002; 346:92-105 and Liu F, Yang J, Huang L, Liu D, New cationic lipid formulations for gene transfer, Pharm. Res. 1996; 13(12):1856-60, which are incorporated by reference.

In addition, the following references discussed in the foregoing examples are incorporated reference:

Arnold J. T., Isaacs J. T. Mechanisms involved in the progression of androgen-independent prostate cancers: it is not only the cancer cell's fault. Endocr. Relat. Cancer. 2002; 9(1):61-73.

Bello, D., M. M. Webber. H. K. Kleinman, D. D. Wartinger, and J. S. Rhim. Androgen responsive adult human prostatic epithelial cell lines immortalized by human papillomavirus 18. Carcinogenesis. 1997; 18:1215-1223.

Berns, K. I., and C. Giraud. Adeno-associated virus (AAV) vectors in gene therapy. Springer, Berlin. 1996; New York.

Boise, L. H., M. Gonzalez-Garcia, C. E. Postema, L. Ding, T. Lindsten, L. A. Turka, X. Mao, G. Nunez, and C. B. Thompson. bcl-x, a bcl-2-related gene that functions as a dominant regulator of apoptotic cell death. 1993; Cell 74:597-608.

Bridge, A. J., S. Pebernard, A. Ducraux, A. L. Nicoulaz, and R. Iggo. Induction of an interferon response by RNAi vectors in mammalian cells. Nat. Genet. 2003; 34:263-264.

Brummelkamp, T. R., Bernards, R, and Agami, R. A system for stable expression of short interfering RNAs in mammalian cells. Science 2002, 296:550-553.

Carson, J. P., G. Kulik, and M. J. Weber. Antiapoptotic signaling in LNCaP prostate cancer cells: a survival signaling pathway independent of phosphatidylinositol 3′-kinase and Akt/protein kinase B. Cancer Res. 1999; 59:1449-1453.

Chen C. D., Sawyers CL. NF-kappa B activates prostate-specific antigen expression and is upregulated in androgen-independent prostate cancer. Mol. Cell Biol. 2002; 22(8):2862-70.

Chen, C. D., D. S. Welsbie, C. Tran, S. H. Baek, R. Chen, R. Vessella, M. G. Rosenfeld, and C. L. Sawyers. Molecular determinants of resistance to antiandrogen therapy. Nat. Med. 2004; 10:33-39.

Claessens, F., G. Verrijdt, E. Schoenmakers, A. Haelens, B. Peeters, G. Verhoeven, and W. Rombauts. Selective DNA binding by the androgen receptor as a mechanism for hormone-specific gene regulation. J. Steroid Biochem. Mol. Biol. 2001; 76:23-30.

Craft N, Sawyers, C. L. Mechanistic concepts in androgen-dependence of prostate cancer. Cancer Metastasis Rev. 1998-99; 17(4):421-7.

Craft, N., Shostak, Y., Carey, M., Sawyers, C. L. A mechanism for hormone-independent prostate cancer through modulation of androgen receptor signaling by the HER-2/neu tyrosine kinase. Nat Med. 1999; 5(3):280-5.

Craft, N., and C. L. Sawyers. Mechanistic concepts in androgen-dependence of prostate cancer. Cancer Metastasis Rev. 1999; 17:421-427.

Csapo, Z., Brand, K., Walther, R., Fokas, K. Comparative experimental study of the serum prostate specific antigen and prostatic acid phosphatase in serially transplantable human prostatic carcinoma lines in nude mice. J. Urol. 1988; 140(5):1032-8.

Culig, Z., Hobisch, A., Cronauer, M. V., Radmayr, C., Trapman, J., Hittmair, A., Bartsch, G., Klocker, H. Androgen receptor activation in prostatic tumor cell lines by insulin-like growth factor-I, keratinocyte growth factor, and epidermal growth factor. Cancer Res. 1994; 54(20):5474-8.

Culig, Z., H. Klocker, G. Bartsch, and A. Hobisch. Androgen receptors in prostate cancer. Endocr. Relat. Cancer. 2002; 9:155-170.

Danial, N. N., and S. J. Korsmeyer. Cell death: critical control points. Cell 2004; 116:205-219.

Denmeade, S. R., Lin X. S. and Isaacs J. T. Role of programmed (apoptotic) cell death during the progression and therapy for prostate cancer. Prostate. 1996; 28(4):251-65.

Devos, A., F. Claessens, P. Alen, J. Winderickx, W. Heyns, W. Rombauts, and B. Peeters. Identification of a functional androgen-response element in the exon 1-coding sequence of the cystatin-related protein gene crp2. Mol Endocrinol. 1997; 11: 1033-1043.

Dou, Q. P. and Li, B. Proteasome inhibitors as potential anticancer agent. Drug Resistant Updates. 1999; 2:215-233.

Duan, D., Yue, Y., Yan, Z., Engelhardt, J. F.: “Trans-splicing Vectors Expand the Packaging Limits of Adeno-associated Virus for Gene Therapy Applications” in Methods in Molecular Medicine. Viral Vectors for Gene Therapy Methods and Protocols Humana Press Inc. Ed: Curtis A. Machida. 2002; 76:287-307.

Duan, D., Fisher, K. J., Burda, J. F., and Engelhardt, J. F. Structural and functional heterogeneity of integrated recombinant AAV genomes. Virus Res. 1997; 48, 41-56.

Duan, D., Sharma, P., Yang, J., Yue, Y., Dudus, L., Zhang, Y., Fisher, K. J., and Engelhardt, J. F. Circular Intermediates of Recombinant Adeno-Associated Virus have Defined Structural Characteristics Responsible for Long Term Episomal Persistence In Muscle. J. Virol. 1998; 72, 8568-8577.

Eder, I. E., Culig, Z., Ramoner, R., Thurnher, M., Putz, T., Nessler-Menardi, C., Tiefenthaler, M., Bartsch, G., and Klocker, H. Inhibition of LncaP prostate cancer cells by means of androgen receptor antisense oligonucleotides. Cancer Gene Ther. 2000; 7: 997-1007.

Eder, I. E., Hoffmann, J., Rogatsch, H., Schafer, G., Zopf, D., Bartsch, G., and Klocker, H. Inhibition of LNCaP prostate tumor growth in vivo by an antisense oligonucleotide directed against the human androgen receptor. Cancer Gene Ther. 2002; 9: 117-125.

El Etreby, M. F., Liang Y, Johnson, M. H., Lewis, R. W. Antitumor activity of mifepristone in the human LNCaP, LNCaP-C4, and LNCaP-C4-2 prostate cancer models in nude mice. Prostate. 2000; 42(2):99-106.

Elbashir, S. M., Harborth, J., Lendeckel, W., Yalcin, A., Weber, K., Tuschl, T. Duplexes of 21-nucleotide RNAs mediate RNA interference in cultured mammalian cells. Nature. 2001; 411(6836):494-8.

Elbashir, S. M., Harborth, J., Weber, K., Tuschl, T. Analysis of gene function in somatic mammalian cells using small interfering RNAs. Methods. 2002; 26(2):199-213.

Gascoyne, D. M., R. M. Kypta, and M. M. Vivanco. Glucocorticoids inhibit apoptosis during fibrosarcoma development by transcriptionally activating Bcl-xL. J. Biol. Chem. 2003; 278:18022-18029.

Gleave M. E., Sato N., Sadar M, Yago V., Bruchovsky N. and Sullivan L. Butyrate analogue, isobutyramide, inhibits tumor growth and time to androgen-independent progression in the human prostate LNCaP tumor model. J. Cell Biochem. 1998; 69(3):271-81.

Goffin F, C. Munaut, F. Frankenne, S. Perrier D'Hauterive, A. Beliard, V. Fridman, P. Nervo, A. Colige, and J. M. Foidart. Expression pattern of metalloproteinases and tissue inhibitors of matrix-metalloproteinases in cycling human endometrium. Biol. Reprod. 2003; 69:976-984.

Gonzalez-Garcia, M., R. Perez-Ballestero, L. Ding, L. Duan, L. H. Boise, C. B. Thompson, and G. Nunez. bcl-x_{L} is the major bcl-x mRNA form expressed during murine development and its product localizes to mitochondria. Development. 1994; 120:3033-3042.

Grad, J. M., X.R. Zeng, and L. H. Boise. Regulation of Bcl-xL: a little bit of this and a little bit of STAT. Curr. Opin. Oncol. 2000; 12:543-549.

Grillot, D. A., M. Gonzalez-Garcia, D. Ekhterae, L. Duan, N. Inohara, S. Ohta, M. F. Seldin, and G. Nunez. Genomic organization, promoter region analysis, and chromosome localization of the mouse bcl-x gene. J. Immunol. 1997; 158:4750-4757.

Gross, A., McDonnell, J. M., and Korsmeyer, S. J. BCL-2 family members and the mitochondria in apoptosis. Genes Dev. 1999; 13: 1899-1911.

Grossmann, M. E., Huang H., Tindall, D. J. Androgen receptor signaling in androgen-refractory prostate cancer. J Natl. Cancer Inst. 2001; 93(22):1687-97.

Hannon G. J. RNA interference. Nature. 2002; 418(6894):244-51.

Harborth, J., Elbashir, S. M., Bechert, K., Tuschl, T., Weber, K. Identification of essential genes in cultured mammalian cells using small interfering RNAs. J Cell Sci. 2001; 114(Pt 24):4557-65.

Hengartner, M. O. The biochemistry of apoptosis. Nature. 2000; 407:770-776.

Holzbeierlein, J., P. Lal, E. LaTulippe, A. Smith, J. Satagopan, L. Zhang, C. Ryan, S. Smith, H. Scher, P. Scardino, V. Reuter, and W. L. Gerald. Gene expression analysis of human prostate carcinoma during hormonal therapy identifies androgen-responsive genes and mechanisms of therapy resistance. Am. J. Pathol. 2004; 164:217-227.

Horoszewicz J. S., Leong S. S., Kawinski E., Karr J. P., Rosenthal H., Chu T. M., Mirand E. A., Murphy G. P. LNCaP model of human prostatic carcinoma. Cancer Res. 1983; 43(4):1809-18.

Huggins C. and Hodges C. V. Studies on prostatic cancer: The effects of castration, of estrogen and of androgen injection on serum phosphatases in metastatic carcinoma of the prostate. Cancer Research. 1941; 1:293-297.

Jemal A., Murray T., Samuels A., Ghafoor A., Ward E., Thun M. J. Cancer statistics, CA Cancer J. Clin. 2003; 53(1):5-26.

Jemal, A, R. C. Tiwari, T. Murray, A. Ghafoor, A. Samuels, E. Ward, E. J. Feuer, M. J. Thun. Cancer statistics. CA Cancer J. Clin. 2004. ;54:8-29.

Kanamaru, H., Li, B., Miwa, Y., Akino, H. and Okada, K. Immunohistochemical expression of p53 and bcl-2proteins is not associated with sarcomatoid change in renal cell carcinoma. Urol. Res. 1998; 27:169-173.

Kay, M. A., C. S. Manno, M. V. Ragni, P. J. Larson, L. B. Couto, A. McClelland, B. Glader, A. J. Chew, S. J. Tai, R. W. Herzog, V. Arruda, F. Johnson, C. Scallan, E. Skarsgard, A. W. Flake, and K. A. High. Evidence for gene transfer and expression of factor IX in haemophilia B patients treated with an AAV vector. Nat Genet. 2000; 24:257-61.

Kelly W. K., Scher H. I. Prostate specific antigen decline after antiandrogen withdrawal: the flutamide withdrawal syndrome. J Urol. 1993; 149(3):607-9.

Kerr, J. F. R., and J. Searle. Deletion of cells by apoptosis during castration-induced involution of the rat prostate. Virchows Arch. B. Cell Pathol. 1973; 13:87-102.

Kerr, J. F. R., and Searle, J. Deletion of cells by apoptosis during castration-induced involution of the rat prostate. Virchows Arch. B. Cell Pathology. 1977; 13: 87.

Kessler, P. D., G. M. Podsakoff, X. Chen, S. A. McQuiston, P. C. Colosi, L. A.Matelis, G. J. Kurtzman, and B. J. Byrne. Gene delivery to skeletal muscle results in sustained expression and systemic delivery of a therapeutic protein. Proc. Natl. Acad. Sci. USA. 1996; 93:14082-7.

Kimura, K., M. Markowski, C. Bowen, and E. P. Gelmann. Androgen blocks apoptosis of hormone-dependent prostate cancer cells. Cancer Res. 2001; 61:5611-5618.

Klein K. A., Reiter R. E., Redula J., Moradi H., Zhu X. L., Brothman A. R., Lamb D. J., Marcelli M., Belldegrun A., Witte O. N., Sawyers C. L. Progression of metastatic human prostate cancer to androgen independence in immunodeficient SCID mice. Nat. Med. 1997; 3(4):402-8.

Kotin, R. M., Linden, R. M., Berns, K. I. Characterization of a preferred site on human chromosome 19q for integration of adeno-associated virus DNA by non-homologous recombination. EMBO J. 1992; 1 1(13):5071-8.

Krajewska, M., S. Krajewski, J. I. Epstein, A. Shabaik, J. Sauvageot, K. Song, S. Kitada, and J. C. Reed. Immunohistochemical analysis of bcl-2, bax, bcl-X, and mcl-1 expression in prostate cancers. Am. J. Pathol. 1996; 148:1567-1576.

Latil, A., I Bieche, D. Vidaud, R. Lidereau, P. Berthon, O. Cussenot, M. Vidaud. Evaluation of androgen, estrogen (ER alpha and ER beta), and progesterone receptor expression in human prostate cancer by real-time quantitative reverse transcription-polymerase chain reaction assays. Cancer Res. 2001; 61:1919-1926.

Lee, N. S., Dohjima, T., Bauer, G., Li H., Li, M. -J., Ehsani, A., Salvaterra, P., Rossi, J. Expression of small interfering RNAs targeted against HIV-1 rev transcripts in human cells. Nature Biotechnology. 2001; 19: 500-505.

Li, B. and Q. P. Dou. Bax degradation by the ubiquitin/proteasome pathway: involvement in tumor survival and progression. Proc. Natl. Acad. Sci. USA. 2000; 97:3850-3855.

Li, B., Desai, S. A., MacCorkle-Chosnek, R. A., Fan, L., Spencer, D. M. A novel conditional Akt ‘survival switch’ reversibly protects cells from apoptosis. Gene Ther. 2002; 9(4):233-44.

Li, B., Kanamaru, H., Noriki, S., Fukuda, M. and Okada, K. Different expression of hepatocyte growth factor in papillary and nodular tumors of the bladder. Int. J. Urol. 1998; 5:436-440.

Li, B., Kanamaru, H., Noriki, S., Fukuda, M. and Okada, K. Numerical aberration of chromosome 17 is strongly correlated with p53 expression, tumor proliferation and histopathology. Int. J. Urol. 1998; 5:317-323.

Li, B., Kanamaru, H., Noriki, S., Yamaguchi, T., Fukuda, M. and Okada, K. Reciprocal expression of bcl-2 and p53 oncoproteins in urothelial dysplasia and carcinoma of the urinary bladder. Urol. Res. 1998; 26:235-241.

Li, B., Saikawa S., Fukuda, M. and Okada, K. Increased cell proliferation of urothelium in rat bladder following unilateral nephrotectomy. J. Urol. 1997; 158:265.

Li, B., and Q. P. Dou. Bax degradation by the ubiquitin/proteasome-dependent pathway: involvement in tumor survival and progression. Proc. Natl. Acad. Sci. USA. 2000; 97:3850-3855.

Li, F., A. Srinivasan, Y. Wang, R. C. Armstrong, K. J. Tomaselli, and L. C. Fritz. Cell-specific induction of apoptosis by microinjection of cytochrome c. Bcl-xL has activity independent of cytochrome c release. J. Biol. Chem. 1997; 272:30299-30305.

Li, P., D. Nijhawan, and X. Wang. Mitochondrial activation of apoptosis. Cell. 2004; 116:S57-59.

Li, P., H. Lee, S. Guo, T. G. Unterman, G. Jenster, and W. Bai. AKT-independent protection of prostate cancer cells from apoptosis mediated through complex formation between the androgen receptor and FKHR. Mol. Cell Biol. 2003; 23:104-118.

Liao, X., J. B. Thrasher, J. Pelling, J. Holzbeierlein, Q. X. Sang, and B. Li. Androgen stimulates matrix metalloproteinase-2 expression in human prostate cancer. Endocrinology. 2003; 144:1656-1663.

Liao, X., J. B. Thrasher, J. Holzbeierlein, S. Stanley, and B. Li. Glycogen synthase kinase-3beta activity is required for androgen-stimulated gene expression in prostate cancer. Endocrinology. 2004; 145:2941-2949.

Liao, X., L. Zhang, J. B. Thrasher, J. Du, and B. Li. GSK-3β suppression eliminates TRAIL-resistance in prostate cancer. Mol. Cancer Ther. 2003; 2:1215-1222.

Martin, S. J., Reutelingsperger, C. P., McGahon, A. J., Rader, J. A., van Schie, R. C., LaFace, D. M., and Green, D. R. Early redistribution of plasma membrane phosphatidylserine is a general feature of apoptosis regardless of the initiating stimulus: inhibition by overexpression of Bcl-2 and Abl. J. Exp. Med. 1995; 182: 1545-1556.

McDonnell T. J., Troncoso P., Brisbay S. M., Logothetis C., Chung L. W., Hsieh J. T., Tu S. M., Campbell M. L. Expression of the protooncogene bcl-2 in the prostate and its association with emergence of androgen-independent prostate cancer. Cancer Res. 1992; 52(24):6940-4.

Mercatante, D. R., J. L. Mohler, and R. Kole. Cellular response to an antisense-mediated shift of Bcl-x pre-mRNA splicing and antineoplastic agents. J. Biol. Chem. 2002; 277:49374-49382.

Miyagishi, M. & Taira, K. U6 promoter-driven siRNAs with four uridine 3′ overhangs efficiently suppress targeted gene expression in mammalian cells. Nature Biotechnology. 2002; 19: 497-500.

Nan, B., T. Snabboon, E. Unni, X -J. Yuan, Y. E. Whang, and M. Marcelli. The PTEN tumor suppressor is a negative modulator of androgen receptor transcriptional activity. J. Mol. Endocrinol. 2003; 31:169-183.

Nickerson, T., F. Chang, D. Lorimer, S. P. Smeekens, C. L. Sawyers, and M. Pollak. In vivo progression of LAPC-9 and LNCaP prostate cancer models to androgen independence is associated with increased expression of insulin-like growth factor I (IGF-I) and IGF-I receptor (IGF-IR). Cancer Res. 2001; 61:6276-6280.

Okabe, M., Ikawa, M., Kominami, K., Nakanishi, T., Nishimune Y. ‘Green mice’ as a source of ubiquitous green cells. FEBS Lett. 1997; 407(3):313-9.

Owens, R. A. Second generation adeno-associated virus type 2-based gene therapy systems with the potential for preferential integration into AAVS 1. Curr Gene Ther. 2002; 2(2):145-59.

Ozanne, D. M., Brady, M. E., Cook S., Gaughan L., Neal D. E., Robson C. N. Androgen receptor nuclear translocation is facilitated by the f-actin cross-linking protein filamin. Mol Endocrinol. 2000; 14(10):1618-26.

Paddison, P. J., Caudy, A. A., Bernstein, E., Hannon, G. J., Conklin, D. S. Short hairpin RNAs (shRNAs) induce sequence-specific silencing in mammalian cells. Genes Dev. 2002; 16(8):948-58.

Paddison, P. J., Caudy, A. A., Hannon G. J. Stable suppression of gene expression by RNAi in mammalian cells. Proc Natl Acad Sci USA. 2002; 99(3):1443-8.

Paez, J. and W. R. Sellers. PI3K/PTEN/AKT pathway. A critical mediator of oncogenic signaling. Cancer Treat. Res. 2003; 115:145-167.

Parrizas, M., and D. LeRoith. Insulin-like growth factor-1 inhibition of apoptosis is associated with increased expression of the bcl-xL gene product. Endocrinology 1997; 138:1355-1358.

Passaniti, A., Isaacs, J. T., Haney, J. A., Adler, S. W., Cujdik, T. J., Long, P. V. and Kleinman, H. K . Stimulation of human prostatic carcinoma tumor growth in athymic mice and control of migration in culture by extracellular matrix. Int. J. Cancer. 1992; 51(2):318-24.

Paul, C. P., Good, P. D., Winer, I., Engelke, D. R. Effective Expression of Small Interfering RNA in human cells. Nature Biotechnology 2002; 19: 505-508.

Paul, R., Breul, J. Antiandrogen withdrawal syndrome associated with prostate cancer therapies: incidence and clinical significance. Drug Saf. 2000; 23(5):381-90.

Petit, P. X., H. Lecoeur, E. Zorn, C. Dauguet, B. Mignotte, and M. L. Gougeon. Alterations in mitochondrial structure and function are early events of dexamethasone-induced thymocyte apoptosis. J. Cell Biol. 1995; 130:157-167.

Raffo, A. J., Perlman, H., Chen, M. W., Day, M. L., Streitman, J. S., Buttyan, R. Overexpression of bcl-2 protects prostate cancer cells from apoptosis in vitro and confers resistance to androgen depletion in vivo. Cancer Res. 1995; 55(19):4438-45.

Saikawa, H., Kanamaru, H., Li, B., Matsukawa, S. and Okada, K. Promoting effect of unilateral nephrotectomy on urinary bladder carcinogenesis in rats. Jpn J. Cancer Res. 1996; 87:1121.

Samulski, R. J., Chang, L. S., Shenk, T. A recombinant plasmid from which an infectious adeno-associated virus genome can be excised in vitro and its use to study viral replication. J. Virol. 1987; 61(10):3096-101.

Scher, H. I., Kelly, W. K. Flutamide withdrawal syndrome: its impact on clinical trials in hormone-refractory prostate cancer. J. Clin. Oncol. 1993; 11 (8):1566-72.

Shariat, S. F., E. Gottenger, C. Nguyen, W. Song, M. W. Kattan, J. Andenoro, T. M. Wheeler, D. M. Spencer, and K. M. Slawin. Preoperative blood reverse transcriptase-PCR assays for prostate-specific antigen and human glandular kallikrein for prediction of prostate cancer progression after radical prostatectomy. Cancer Res. 2002; 62:5974-5979.

Sledz, C.A., M. Holko, M. J. de Veer, R. H. Silverman, and B. R. Williams. Activation of the interferon system by short-interfering RNAs. Nat. Cell Biol. 2003; 5:834-839.

Soto, A. M., Lin, T.M., Sakabe, K., Olea, N., Damassa, D. A., Sonnenschein, C. Variants of the human prostate LNCaP cell line as tools to study discrete components of the androgen-mediated proliferative response. Oncol Res. 1995; 7(10-11):545-58.

Spanaus, K. S., R. Schlapbach, and A. Fontana. TNF-alpha and IFN-gamma render microglia sensitive to Fas ligand-induced apoptosis by induction of Fas expression and down-regulation of Bcl-2 and Bcl-xL. Eur. J. Immunol. 1998; 28:4398-4408.

Sramkoski, R. M., T. G. 2^{nd}. Pretlow, J. M. Giaconia, T. P. Pretlow, S. Schwartz, M. S. Sy, S. R. Marengo, J. S. Rhim, D. Zhang, and J. W. Jacobberger. A new human prostate carcinoma cell line, 22Rv1. In Vitro Cell Dev. Biol. Anim. 1999; 35:403-409.

Stanbrough, M., I. Leav, P.W. Kwan, G. J. Bubley, and S. P. Balk. Prostatic intraepithelial neoplasia in mice expressing an androgen receptor transgene in prostate epithelium. Proc. Natl. Acad. Sci. U S A. 2001; 98:10823-10828.

Stedman, H., J. M. Wilson, R. Finke, A. L. Kleckner, and J. Mendell. Phase I clinical trial utilizing gene therapy for limb girdle muscular dystrophy: alpha-, beta-, gamma-, or delta-sarcoglycan gene delivered with intramuscular instillations of adeno-associated vectors. Hum Gene Ther. 2000; 11:777-90.

Sui, G., Soohoo, C., Affar, E. B., Gay, F., Shi, Y., Forrester, W. C., and Shi, Y. A DNA vector-based RNAi technology to suppress gene expression in mammalian cells. Proc Natl Acad Sci USA. 2002; 99(8): 5515-5520.

Sun, J. Z., Nam, S., Lee, C. S., Li, B., Coppola, D., Hamilton, A. D., Dou, Q. P. and Sebti, S.M. CEP1612, a dipeptidyl proteasome inhibitor, induces p21^{waf1} and p27^{kip1} expression and apoptosis and inhibits the growth of the human lung adenocarcinoma A-549 in nude mice. Cancer Res. 2000; 61:1280-1284.

Taplin, M. E., and S. P. Balk. Androgen receptor: a key molecule in the progression of prostate cancer to hormone independence. J. Cell Biochem. 2004; 91:483-490.

Thalmann, G. N., Anezinis, P. E., Chang, S.M., Zhau, H. E., Kim, E. E., Hopwood, V. L., Pathak, S., von Eschenbach, A. C., Chung, L. W. Androgen-independent cancer progression and bone metastasis in the LNCaP model of human prostate cancer. Cancer Res. 1994; 54(10):2577-81.

Tijsterman, M., and R. H. Plasterk. Dicers at RISC; the mechanism of RNAi. Cell. 2004; 117:1-3.

Tosetti, F., Vene, R., Arena, G., Morini, M., Minghelli, S., Noonan, D. M., Albini, A. N-(4-Hydroxyphenyl) retinamide Inhibits Retinoblastoma Growth through Reactive Oxygen Species-Mediated Cell Death. Mol Pharmacol. 2003; 63(3):565-573.

Tsuka H, Mori H Benyi Li, Kanamaru H, Matsukawa S and Okada K. Expression of c-Met/HGF receptor mRNA and protein in human non-malignant and malignant prostate tissue. Int. J. Oncol. 1998; 13:927-934.

Tsuka, H., Mori, H., Benyi Li, Kanamaru, H., Matsukawa, .S. and Okada, K. Enhanced hepatocyte growth factor level in human prostate cancer treated with endocrine therapy. Int. J. Oncol. 1998; 13:169-176.

van Steenbrugge G. J., van Uffelen C. J., Bolt J., and Schroder F. H. The human prostatic cancer cell line LNCaP and its derived sublines: an in vitro model for the study of androgen sensitivity. J. Steroid. Biochem. Mol. Biol. 1991; 40: 207-214.

Viegas, L. R., Vicent, G. P., Baranao, J. L., Beato, M., and Pecci, A. Steroid hormones induce bcl-X gene expression through direct activation of distal promoter P4. J. Biol. Chem. 2004; 279:9831-9839.

Vlietstra, R. J., D. C. van Alewijk, K.G. Hermans, G. J. van Steenbrugge, and J.Trapman. Frequent inactivation of PTEN in prostate cancer cell lines and xenografts. Cancer Res. 1998; 58:2720-2723.

Wagner, J. A., T. Reynolds, M. L. Moran, R. B. Moss, J. J. Wine, T. R. Flotte, and P. Gardner. Efficient and persistent gene transfer of AAV-CFTR in maxillary sinus [letter]. Lancet. 1998; 351:1702-3.

Wen, Y., M. C. Hu, K. Makino, B. Spohn, G. Bartholomeusz, D. H. Yan, and M. C. Hung. HER-2/neu promotes androgen-independent survival and growth of prostate cancer cells through the Akt pathway. Cancer Res. 2000; 60:6841-6845.

Wright, M. E., Tsai, M. J., and Aebersold, R. Androgen receptor represses the neuroendocrine transdifferentiation process in prostate cancer cells. Mol. Endocrinol. 2003; 17: 1726-1737.

Wu, H. C., Hsieh, J. T., Gleave, M. E., Brown, N. M., Pathak, S., and Chung, L. W. Derivation of androgen-independent human LNCaP prostatic cancer cell sublines: role of bone stromal cells. Int. J. Cancer. 1994; 57: 406-412.

Xia H, Mao Q, Paulson H L, Davidson B L. siRNA-mediated gene silencing in vitro and in vivo. Nat Biotechnol. 2002; 20(10):1006-10.

Xiao, X., J. Li, and R. J. Samulski. Efficient long-term gene transfer into muscle tissue of immunocompetent mice by adeno-associated virus vector. J Virol. 1996; 70:8098-108.

Xiao, X., Li, J., and Samulski, R. J. Production of high-titer recombinant adeno-associated virus vectors in the absence of helper adenovirus. J. Virol. 1998; 72, 2224-32.

Xie X, Zhao X, Liu Y, Zhang J, Matusik R J, Slawin K M, Spencer D M. Adenovirus-mediated tissue-targeted expression of a caspase-9-based artificial death switch for the treatment of prostate cancer. Cancer Res. 2001; 61(18):6795-804.

Yu, J. Y., DeRuiter, S. L., Turner, D. L. RNA interference by expression of short-interfering RNAs and hairpin RNAs in mammalian cells. Proc. Natl. Acad. Sci. USA. 2002; 99(9):6047-52.

Zegarra-Moro, O. L., Schmidt, L. J., Huang, H., Tindall, D. J. Disruption of androgen receptor function inhibits proliferation of androgen-refractory prostate cancer cells. Cancer Res. 2002; 62(4):1008-13.

Zhang, L., Johnson, M., Le, K. H., Sato, M., Ilagan, R., Iyer, M., Gambhir, S. S., Wu, L., Carey, M. Interrogating androgen receptor function in recurrent prostate cancer. Cancer Res. 2003; 63: 4552-4560.

While specific embodiments have been shown and discussed, various modifications may of course be made, and the invention is not limited to the specific forms or arrangement of steps described herein, except insofar as such limitations are included in the following claims. Further, it will be understood that certain features and sub-combinations are of utility and may be employed without reference to other features and sub-combinations. This is contemplated by and is within the scope of the claims.

1. A short interfering nucleic acid (siRNA) molecule that down regulates expression of an androgen receptor (AR) gene in a cell by RNA interference and induces apoptosis therein.

2. The siRNA molecule of claim 1, wherein said siRNA molecule is adapted for use to treat prostate cancer.

3. The siRNA molecule of claim 1, wherein said siRNA molecule comprises a sense region and an antisense region and wherein said antisense region comprises sequence complementary to an RNA sequence encoding the AR and the sense region comprises sequence complementary to the antisense region.

4. The siRNA molecule of claim 3, wherein said siRNA molecule is assembled from two nucleic acid fragments wherein one fragment comprises the sense region and the second fragment comprises the antisense region of said siRNA molecule.

5. The siRNA molecule of claim 4, wherein said sense region and antisense region are covalently connected via a linker molecule.

6. The siRNA molecule of claim 5, wherein said linker molecule is a polynucleotide linker.

7. The siRNA molecule of claim 5, wherein said linker molecule is a non-nucleotide linker.

8. The siRNA molecule of claim 3, wherein said antisense region comprises sequence complementary to sequence having SEQ. ID NO. 8.

9. The siRNA molecule of claim 3, wherein said antisense region comprises sequence having any of SEQ ID NO. 8 and SEQ. ID NO. 31.

10. An expression vector comprising a nucleic acid sequence encoding at least one siRNA molecule of claim 1 in a manner that allows expression of the nucleic acid molecule.

11. The expression vector of claim 10, wherein said siRNA molecule comprises a sense region and an antisense region and wherein said antisense region comprises sequence complementary to an RNA sequence encoding AR and the sense region comprises sequence complementary to the antisense region.

12. The expression vector of claim 10, wherein said siRNA molecule comprises two distinct strands having complementarity sense and antisense regions.

13. The expression vector of claim 10, wherein said siRNA molecule comprises a single strand having complementary sense and antisense regions.

14. A mammalian cell comprising an expression vector of claim 10.

15. The mammalian cell of claim 10, wherein said mammalian cell is a human cell.

16. A recombinant plasma comprising nucleic acid sequences for expression the siRNA of claim 1.

17. The recombinant plasmid of claim 16, wherein the nucleic acid sequences for expressing the siRNA comprise an inducible or regulatable promoter.

18. The recombinant plasmid of claim 16, wherein the plasmid comprises a CMV promoter.

19. A recombinant viral vector comprising nucleic acid sequences for expressing the siRNA molecule of claim 1.

20. The recombinant viral vector of claim 19, wherein the nucleic acid sequences for expressing the siRNA comprise an inducible or regulatable promoter.

21. The recombinant viral vector of claim 19, wherein the recombinant viral vector is an adeno-associated viral vector

22. A method for inhibiting the growth of a prostate cancerous cell population comprising:
applying the siRNA of claim 1 to said cancerous cell population.

23. The method of claim 22 wherein said cell population undergoes apoptosis.

24. The method of claim 23 wherein said apoptosis is evidenced by PARP cleavage.

25. The method of claim 23 wherein said apoptosis is mediated by reducing Bcl-xL expression.

26. The method of claim 22 wherein said cancerous cell population is a human prostate cancerous cell population.

27. A method to inhibit expression of an androgen receptor gene in a prostate cancer cell in vitro comprising introduction of a ribonucleic acid (RNA) into the cell in an amount sufficient to inhibit expression of the target gene, wherein the RNA is a double-stranded molecule with a first strand consisting essentially of a ribonucleotide sequence which corresponds to a nucleotide sequence of the target gene and a second strand consisting essentially of a ribonucleotide sequence which is complementary to the nucleotide sequence of the target gene, wherein the first and the second ribonucleotide strands are separate complementary strands that hybridize to each other to form said double-stranded molecule, and the double-stranded molecule inhibits expression.

28. The method of claim 27 in which the first ribonucleotide sequence comprises at least 19 bases which correspond to the target gene and the second ribonucleotide sequence comprises at least 19 bases which are complementary to the nucleotide sequence of the target gene.

29. The method of claim 27 in which the prostate cancer cell is an androgen-sensitive cell.

30. The method of claim 27 wherein said prostate cancerous cell population is selected from the group consisting of LNCaP and PC-3 cell populations.


SEQUENCE LISTING (50 sequences)

SEQ ID NO: 1 - RNA, 19 nt, Homo sapiens
  ref <308>: DATABASE ACCESSION NUMBER: GenBank/NM_000044
  ref <309>: DATABASE ENTRY DATE: 2002-06-04
  ref <313>: RELEVANT RESIDUES: (577)..(595)
cuccuucagcaacagcagc

SEQ ID NO: 2 - RNA, 19 nt, Homo sapiens
  ref <308>: DATABASE ACCESSION NUMBER: GenBank/NM_000044
  ref <309>: DATABASE ENTRY DATE: 2002-06-04
  ref <313>: RELEVANT RESIDUES: (589)..(607)
cagcagcaggaagcaguau

SEQ ID NO: 3 - RNA, 19 nt, Homo sapiens
  ref <308>: DATABASE ACCESSION NUMBER: GenBank/NM_000044
  ref <309>: DATABASE ENTRY DATE: 2002-06-04
  ref <313>: RELEVANT RESIDUES: (601)..(619)
gcaguauccgaaggcagca

SEQ ID NO: 4 - RNA, 19 nt, Homo sapiens
  ref <308>: DATABASE ACCESSION NUMBER: GenBank/NM_000044
  ref <309>: DATABASE ENTRY DATE: 2002-06-04
  ref <313>: RELEVANT RESIDUES: (705)..(723)
cgccaaggaguuguguaag

SEQ ID NO: 5 - RNA, 19 nt, Homo sapiens
  ref <308>: DATABASE ACCESSION NUMBER: GenBank/NM_000044
  ref <309>: DATABASE ENTRY DATE: 2002-06-04
  ref <313>: RELEVANT RESIDUES: (711)..(729)
ggaguuguguaaggcagug

SEQ ID NO: 6 - RNA, 19 nt, Homo sapiens
  ref <308>: DATABASE ACCESSION NUMBER: GenBank/NM_000044
  ref <309>: DATABASE ENTRY DATE: 2002-06-04
  ref <313>: RELEVANT RESIDUES: (873)..(891)
agguucucugcuagacgac

SEQ ID NO: 7 - RNA, 19 nt, Homo sapiens
  ref <308>: DATABASE ACCESSION NUMBER: GenBank/NM_000044
  ref <309>: DATABASE ENTRY DATE: 2002-06-04
  ref <313>: RELEVANT RESIDUES: (874)..(892)
gguucucugcuagacgaca

SEQ ID NO: 8 - RNA, 19 nt, Homo sapiens
  ref <308>: DATABASE ACCESSION NUMBER: GenBank/NM_000044
  ref <309>: DATABASE ENTRY DATE: 2002-06-04
  ref <313>: RELEVANT RESIDUES: (1324)..(1342)
gaaggccaguuguauggac

SEQ ID NO: 9 - RNA, 19 nt, Homo sapiens
  ref <308>: DATABASE ACCESSION NUMBER: GenBank/NM_000044
  ref <309>: DATABASE ENTRY DATE: 2002-06-04
  ref <313>: RELEVANT RESIDUES: (1330)..(1348)
ggccaguuguauggaccgu

SEQ ID NO: 10 - RNA, 19 nt, Homo sapiens
  ref <308>: DATABASE ACCESSION NUMBER: GenBank/NM_000044
  ref <309>: DATABASE ENTRY DATE: 2002-06-04
  ref <313>: RELEVANT RESIDUES: (1674)..(1692)
gaccugccugagcugugga

SEQ ID NO: 11 - RNA, 19 nt, Homo sapiens
  ref <308>: DATABASE ACCESSION NUMBER: GenBank/NM_000044
  ref <309>: DATABASE ENTRY DATE: 2002-06-04
  ref <313>: RELEVANT RESIDUES: (1773)..(1791)
acagaaguaccugugcgcc

SEQ ID NO: 12 - RNA, 19 nt, Homo sapiens
  ref <308>: DATABASE ACCESSION NUMBER: GenBank/NM_000044
  ref <309>: DATABASE ENTRY DATE: 2002-06-04
  ref <313>: RELEVANT RESIDUES: (1774)..(1792)
cagaaguaccugugcgcca

SEQ ID NO: 13 - RNA, 19 nt, Homo sapiens
  ref <308>: DATABASE ACCESSION NUMBER: GenBank/NM_000044
  ref <309>: DATABASE ENTRY DATE: 2002-06-04
  ref <313>: RELEVANT RESIDUES: (1917)..(1935)
acuacaggaggaaggagag

SEQ ID NO: 14 - RNA, 19 nt, Homo sapiens
  ref <308>: DATABASE ACCESSION NUMBER: GenBank/NM_000044
  ref <309>: DATABASE ENTRY DATE: 2002-06-04
  ref <313>: RELEVANT RESIDUES: (1918)..(1936)
cuacaggaggaaggagagg

SEQ ID NO: 15 - RNA, 19 nt, Homo sapiens
  ref <308>: DATABASE ACCESSION NUMBER: GenBank/NM_000044
  ref <309>: DATABASE ENTRY DATE: 2002-06-04
  ref <313>: RELEVANT RESIDUES: (1970)..(1988)
cccagaagcugacaguguc

SEQ ID NO: 16 - RNA, 19 nt, Homo sapiens
  ref <308>: DATABASE ACCESSION NUMBER: GenBank/NM_000044
  ref <309>: DATABASE ENTRY DATE: 2002-06-04
  ref <313>: RELEVANT RESIDUES: (1999)..(2017)
ggcuaugaaugucagccca

SEQ ID NO: 17 - RNA, 19 nt, Homo sapiens
  ref <308>: DATABASE ACCESSION NUMBER: GenBank/NM_000044
  ref <309>: DATABASE ENTRY DATE: 2002-06-04
  ref <313>: RELEVANT RESIDUES: (2028)..(2046)
uguccuggaagccauugag

SEQ ID NO: 18 - RNA, 19 nt, Homo sapiens
  ref <308>: DATABASE ACCESSION NUMBER: GenBank/NM_000044
  ref <309>: DATABASE ENTRY DATE: 2002-06-04
  ref <313>: RELEVANT RESIDUES: (2038)..(2056)
gccauugagccagguguag

SEQ ID NO: 19 - RNA, 19 nt, Homo sapiens
  ref <308>: DATABASE ACCESSION NUMBER: GenBank/NM-000044
  ref <309>: DATABASE ENTRY DATE: 2002-06-04
  ref <313>: RELEVANT RESIDUES: (2076)..(2094)
caaccagcccgacuccuuu

SEQ ID NO: 20 - RNA, 19 nt, Homo sapiens
  ref <308>: DATABASE ACCESSION NUMBER: GenBank/NM_000044
  ref <309>: DATABASE ENTRY DATE: 2002-06-04
  ref <313>: RELEVANT RESIDUES: (2184)..(2202)
cuuacacguggacgaccag

SEQ ID NO: 21 - RNA, 19 nt, Homo sapiens
  ref <308>: DATABASE ACCESSION NUMBER: GenBank/NM_000044
  ref <309>: DATABASE ENTRY DATE: 2002-06-04
  ref <313>: RELEVANT RESIDUES: (2271)..(2289)
ugucaacuccaggaugcuc

SEQ ID NO: 22 - RNA, 19 nt, Homo sapiens
  ref <308>: DATABASE ACCESSION NUMBER: GenBank/NM_000044
  ref <309>: DATABASE ENTRY DATE: 2002-06-04
  ref <313>: RELEVANT RESIDUES: (2277)..(2295)
cuccaggaugcucuacuuc

SEQ ID NO: 23 - RNA, 19 nt, Homo sapiens
  ref <308>: DATABASE ACCESSION NUMBER: GenBank/NM_000044
  ref <309>: DATABASE ENTRY DATE: 2002-06-04
  ref <313>: RELEVANT RESIDUES: (2316)..(2334)
ugaguaccgcaugcacaag

SEQ ID NO: 24 - RNA, 19 nt, Homo sapiens
  ref <308>: DATABASE ACCESSION NUMBER: GenBank/NM_000044
  ref <309>: DATABASE ENTRY DATE: 2002-06-04
  ref <313>: RELEVANT RESIDUES: (2363)..(2381)
ugaggcaccucucucaaga

SEQ ID NO: 25 - RNA, 19 nt, Homo sapiens
  ref <308>: DATABASE ACCESSION NUMBER: GenBank/NM_000044
  ref <309>: DATABASE ENTRY DATE: 2002-06-04
  ref <313>: RELEVANT RESIDUES: (2398)..(2416)
aucaccccccaggaauucc

SEQ ID NO: 26 - RNA, 19 nt, Homo sapiens
  ref <308>: DATABASE ACCESSION NUMBER: GenBank/NM_000044
  ref <309>: DATABASE ENTRY DATE: 2002-06-04
  ref <313>: RELEVANT RESIDUES: (2399)..(2417)
ucaccccccaggaauuccu

SEQ ID NO: 27 - RNA, 19 nt, Homo sapiens
  ref <308>: DATABASE ACCESSION NUMBER: GenBank/NM_000044
  ref <309>: DATABASE ENTRY DATE: 2002-06-04
  ref <313>: RELEVANT RESIDUES: (2427)..(2445)
agcacugcuacucuucagc

SEQ ID NO: 28 - RNA, 19 nt, Homo sapiens
  ref <308>: DATABASE ACCESSION NUMBER: GenBank/NM_000044
  ref <309>: DATABASE ENTRY DATE: 2002-06-04
  ref <313>: RELEVANT RESIDUES: (2428)..(2446)
gcacugcuacucuucagca

SEQ ID NO: 29 - RNA, 19 nt, Homo sapiens
  ref <308>: DATABASE ACCESSION NUMBER: GenBank/NM_000044
  ref <309>: DATABASE ENTRY DATE: 2002-06-04
  ref <313>: RELEVANT RESIDUES: (2548)..(2566)
aucccacauccugcucaag

SEQ ID NO: 30 - RNA, 19 nt, Homo sapiens
  ref <308>: DATABASE ACCESSION NUMBER: GenBank/NM_000044
  ref <309>: DATABASE ENTRY DATE: 2002-06-04
  ref <313>: RELEVANT RESIDUES: (2547)..(2565)
ucccacauccugcucaaga

SEQ ID NO: 31 - RNA, 19 nt, Homo sapiens
  ref <308>: DATABASE ACCESSION NUMBER: GenBank/NM_000044
  ref <309>: DATABASE ENTRY DATE: 2002-06-04
  ref <313>: RELEVANT RESIDUES: (2564)..(2582)
gacgcuucuaccagcucac

SEQ ID NO: 32 - RNA, 19 nt, Homo sapiens
  ref <308>: DATABASE ACCESSION NUMBER: GenBank/NM_000044
  ref <309>: DATABASE ENTRY DATE: 2002-06-04
  ref <313>: RELEVANT RESIDUES: (2652)..(2670)
gucacacauggugagcgug

SEQ ID NO: 33 - RNA, 19 nt, Homo sapiens
  ref <308>: DATABASE ACCESSION NUMBER: GenBank/NM_000044
  ref <309>: DATABASE ENTRY DATE: 2002-06-04
  ref <313>: RELEVANT RESIDUES: (2710)..(2728)
gugcccaugauccuuucug

SEQ ID NO: 34 - RNA, 19 nt, Homo sapiens
  ref <308>: DATABASE ACCESSION NUMBER: GenBank/NM_000044
  ref <309>: DATABASE ENTRY DATE: 2002-06-04
  ref <313>: RELEVANT RESIDUES: (2739)..(2757)
gcccaucuauuuccacacc

SEQ ID NO: 35 - DNA, 21 bp, Homo sapiens
cctggcttccgcaacttacac

SEQ ID NO: 36 - DNA, 22 bp, Homo sapiens
ggacttgtgcatgcggtactca

SEQ ID NO: 37 - DNA, 20 bp, Homo sapiens
gatgactccagccacgacct

SEQ ID NO: 38 - DNA, 20 bp, Homo sapiens
cacagacaccccatcctatc

SEQ ID NO: 39 - DNA, 20 bp, Homo sapiens
catggcagcagtaaagcaag

SEQ ID NO: 40 - DNA, 21 bp, Homo sapiens
gcattgttcccatagagttcc

SEQ ID NO: 41 - DNA, 24 bp, Homo sapiens
gttcacccactaatagggaacgtg

SEQ ID NO: 42 - DNA, 24 bp, Homo sapiens
gattctgacttagaggcgttcagt

SEQ ID NO: 43 - DNA, 20 bp, Homo sapiens
cgatggaggaggaagcaagc

SEQ ID NO: 44 - DNA, 21 bp, Homo sapiens
gcaccacctacattcaaatcc

SEQ ID NO: 45 - RNA, 19 nt, Homo sapiens
gaaucgagagcuccagauc

SEQ ID NO: 46 - DNA, 17 bp, Homo sapiens
tgtgatacaaaagatct

SEQ ID NO: 47 - DNA, 11 bp, Homo sapiens
tgtcgccttct

SEQ ID NO: 48 - DNA, 8 bp, Homo sapiens
tggttcct

SEQ ID NO: 49 - DNA, 25 bp, Homo sapiens
agatgggcttgactttcccagaaag

SEQ ID NO: 50 - DNA, 25 bp, Homo sapiens
atggctgtcattcagtactcctgga